(19) United States
(12) Patent Application Publication
(10) Pub. No.: US 2012/0003160 A1
(43) Pub. Date: Jan. 5, 2012

(54) Macrophage-Enhanced MRI (MEMRI) in a Single Imaging Session

(75) Inventors: Gerald L. Wolf, Newton, MA (US); Jerome M. Lewis, Newton, MA (US)

(73) Assignee: AMAG PHARMACEUTICALS, INC., Lexington, MA (US)

(21) Appl. No.: 13/197,153

(22) Filed: Aug. 3, 2011

Related U.S. Application Data
(63) Continuation-in-part of application No. 12/145,565, filed on Jun. 25, 2008, now abandoned.
(60) Provisional application No. 60/947,259, filed on Jun. 29, 2007.
(60) Provisional application No. 61/370,167, filed on Aug. 3, 2010.

(51) Int. Cl. A61K 49/08

(52) U.S. Cl. 424/9.32; 424/9.3; 424/9.36

(57) ABSTRACT

Methods for performing macrophage-enhanced MRI, utilizing a macrophage imaging agent, in a single imaging session are provided. The macrophage imaging agent may be an ultrasmall superparamagnetic iron oxide particle. One embodiment includes administering a macrophage imaging agent to the subject during an administration session then allowing a passage of time sufficient for accumulation of the agent in macrophages of the subject. Subsequently, in a single imaging session, a macrophage-enhanced magnetic resonance image is acquired to target macrophages and a different magnetic resonance image is acquired to target physiological phenomenon other than macrophages. Additional embodiments provide methods wherein the acquisition of a different magnetic resonance image is achieved by vascular-enhanced MRI protocols or perfusion-enhanced MRI protocols, or combinations thereof. Further embodiments provide methods for utilizing acquired images in assessment of treatment of disease.

### CROSS REFERENCE TO RELATED APPLICATIONS

This application is a continuation-in-part of application Ser. No. 12/145,565 filed Jun. 25, 2008, which in turn claims the benefit of U.S. Provisional Application Ser. No. 60/947,259, filed Jun. 29, 2007, both of which are hereby incorporated by reference herein; this application also claims the benefit of U.S. Provisional Application No. 61/370,167 filed Aug. 3, 2010, which is hereby incorporated by reference herein.

### TECHNICAL FIELD

The present invention relates to whole-body MRI scanning and cancer staging using macrophage-seeking MRI agents to perform Macrophage-Enhanced MRI or “MEMRI”.

### BACKGROUND ART

Cancer is one of the leading causes of death in the developed world, resulting in over 500,000 deaths per year in the United States alone. Over one million people are diagnosed with cancer in the U.S. each year, and overall it is estimated that more than 1 in 3 people will develop some form of cancer during their lifetime. Though there are more than 200 different types of cancer, four of them—breast, lung, colorectal, and prostate—account for over half of all new cases (Jemal et al., CA CANCER J. CLIN. 53:5-26 (2003)). Cancer metastasis is considered to be due to the distribution of cancer cells via the blood—with liver, lung, bone, and CNS as common sites at risk, or the lymphatics with lymph node and bone as metastatic risk sites.

Breast cancer is the most common cancer in women, with an estimate 12% of women at risk of developing the disease during their lifetime. Although mortality rates have decreased due to earlier detection and improved treatments, breast cancer remains a leading cause of death in middle-aged women. Furthermore, metastatic breast cancer is still an incurable disease. On presentation, most patients with metastatic breast cancer have only one or two organ systems affected, but as the disease progresses, multiple sites usually become involved. The most common sites of metastatic involvement are locoregional recurrences in the skin and soft tissues of the chest wall, as well as in regional lymph nodes. The most common site for distant metastasis is the bone (30-40% of distant metastasis), followed by the lungs and liver. And although only approximately 1-5% of women with newly diagnosed breast cancer have distant metastasis at the time of diagnosis, approximately 50% of patients with local disease eventually relapse with metastasis within five years. At present the median survival from the manifestation of distant metastases is about three years.

Current methods of diagnosing and staging breast cancer include the tumor-node-metastasis (“TNM”) system that relies on tumor size, tumor presence in lymph nodes, and the presence of distant metastases as described in the American Joint Committee on Cancer, AJCC Cancer Staging Manual, Philadelphia, Pa., Lippincott-Raven Publishers, 6th ed. (2006), pp 221-240, and in Harris, J R: “Staging of breast carcinoma” in Harris, J. R., et al., eds., BREAST DISEASES, Philadelphia, Lippincott (1991). These parameters are used to provide a prognosis and select an appropriate therapy. The morphologic appearance of the tumor can also be assessed but because tumors with similar histopathologic appearance can exhibit significant clinical variability, this approach has serious limitations. Finally assays for cell surface markers can be used to divide certain tumors types into subclasses. For example, one factor considered in the prognosis and treatment of breast cancer is the presence of the estrogen receptor (ER) as ER-positive breast cancers typically respond more readily to hormonal therapies such as tamoxifen or aromatase inhibitors than ER-negative tumors. Yet these analyses, though useful, are only partially predictive of the clinical behavior of breast tumors, and there is much phenotypic diversity present in breast cancers that current diagnostic tools fail to detect and current therapies fail to treat.

Prostate cancer is the most common cancer in men in the developed world, representing an estimated 33% of all new cancer cases in the U.S., and is the second most frequent cause of death (Jemal et al., CA CANCER J. CLIN. 53:5-26 (2003)). Since the introduction of the prostate specific antigen (PSA) blood test, early detection of prostate cancer has dramatically improved survival rates, and the five year survival rate for patients with local and regional stage prostate cancers at the time of diagnosis is nearing 100%. Yet more than 50% of patients will eventually develop locally advanced or metastatic disease (Muthuramalingam et al., CLIN. ONCOL. 16:505-516 (2004)).

Currently radical prostatectomy and radiation therapy provide curative treatment for the majority of localized prostate tumors. However, therapeutic options are very limited for advanced cases. For metastatic disease, androgen ablation with luteinizing hormone-releasing hormone (LHRH) agonist alone or in combination with anti-androgens is the standard treatment. Yet despite maximal androgen blockage, the disease nearly always progresses with the majority developing androgen-independent disease. At present there is no uniformly accepted treatment for hormone refractory prostate cancer, and chemotherapeutic regimes are commonly used (Muthuramalingam et al., CLIN. ONCOL. 16:505-516 (2004); Trojan et al., ANTICANCER RES. 25:551-561 (2005)).

Colorectal cancer is the third most common cancer and the fourth most frequent cause of cancer deaths worldwide (Weitz et al., 2005, LANCET 365:153-65). Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Colorectal carcinoma has a tendency to invade locally by circumferential growth and elsewhere by lymphatic, hematogenous, transperitoneal, and perineural spread. The most common site of extralymphatic involvement is the liver, with the lungs the most frequently affected extra-abdominal organ. Other sites of hematogenous spread include the bones, kidneys, adrenal glands, and brain.

The current staging system for colorectal cancer is based on the degree of tumor penetration through the bowel wall and the presence or absence of nodal involvement. This staging system is defined by three major Duke's classifications: Duke's A disease is confined to submucosa layers of colon or rectum; Duke's B disease has tumors that invade through the muscularis propria and may penetrate the wall of the colon or rectum; and Duke's C disease includes any degree of bowel wall invasion with regional lymph node metastasis. While surgical resection is highly effective for early stage colorectal cancers, providing cure rates of 95% in Duke's A patients, the rate is reduced to 75% in Duke's B patients and the presence of positive lymph node in Duke's C disease predicts a 60% likelihood of recurrence within five years. Treatment of Duke's C patients with a post surgical course of chemotherapy reduces the recurrence rate to 40%-50%, and is now the standard of care for these patients.

Lung cancer is the most common cancer worldwide, the third most commonly diagnosed cancer in the United States, and by far the most frequent cause of cancer deaths (Spiro et al., AM. J. RESPIR. CRIT. CARE MED. 166:1166-1196 (2002); Jemal et al., CA CANCER J. CLIN. 53:5-26 (2003)). Cigarette smoking is believed responsible for an estimated 87% of all lung cancers making it the most deadly preventable disease. Lung cancer is divided into two major types that account for over 90% of all lung cancers: small cell lung cancer (SCLC) and non-small cell lung cancer (NSCLC). SCLC accounts for 15-20% of cases and is characterized by its origin in large central airways and histological composition of sheets of small cells with little cytoplasm. SCLC is more aggressive than NSCLC, growing rapidly and metastasizing early and often. NSCLC accounts for 80-85% of all cases and is further divided into three major subtypes based on histology: adenocarcinoma, squamous cell carcinoma (epidermoid carcinoma), and large cell undifferentiated carcinoma. The most common metastatic sites are pleura, lung, bone, liver, brain, and pericardium.

Lung cancer typically presents late in its course, and thus has a median survival of only 6-12 months after diagnosis and an overall 5 year survival rate of only 5-10%. Although surgery offers the best chance of a cure, only a small fraction of lung cancer patients are eligible with the majority relying on chemotherapy and radiotherapy. Despite attempts to manipulate the timing and dose intensity of these therapies, survival rates have increased little over the last 15 years (Spiro et al., AM. J. RESPIR. CRIT. CARE MED. 166:1166-1196 (2002)).

Cancer arises from dysregulation of the mechanisms that control normal tissue development and maintenance. Solid tumors are composed of heterogeneous cell populations. For example, breast cancers are a mixture of cancer cells and normal cells, including mesenchymal (stromal) cells, inflammatory cells, and endothelial cells. Classic models of cancer hold that phenotypically distinct cancer cell populations all have the capacity to proliferate and give rise to a new tumor. In the classical model, tumor cell heterogeneity results from environmental factors as well as ongoing mutations within cancer cells resulting in a diverse population of tumorigenic cells. This model rests on the idea that all populations of tumor cells would have some degree of tumorigenic potential. (Pandis et al., GENES, CHROMOSOMES & CANCER 12:122-129 (1998); Kuukasjrvi et al., CANCER RES. 57:1597-1604 (1997); Bonsing et al., CANCER 71:382-391 (1993); Bonsing et al., GENES CHROMOSOMES & CANCER 82:173-183 (2000); Beerman H. et al., CYTOMETRY. 12:147-154 (1991); Aubele M & Werner M, Analyt. CELL. PATH. 19:53 (1999); Shen L et al., CANCER RES. 60:3884 (2000)).

The heterogeneous cell populations include variable numbers of macrophages. These tumor-associated macrophages (“TAMs”) are derived from monocytes circulating in the blood, where they are attracted to the tumor. Although macrophages normally play a vital role in marshalling immune-mediated defenses, TAMs contribute to tumor progression by promoting tumor cell survival, proliferation and metastasis. It is thought that macrophage density reflects tumor aggressiveness; more malignant tumors have more macrophages nearby, whereas benign tumors have few associated TAMs, TAMS may also assist in dissemintation of the tumor.

Like cancer, vascular disease is a leading cause of mortality. Vascular disease can be defined by the area of the vasculature in which it is located. Coronary artery disease (CAD), or coronary heart disease, afflicts over 16 million Americans (see American Heart Association, 2009 Update At-A-Glance, http://www.americanheart.org/downloadable/heart/1240250946756LS-1982%20Heart%20and%20Stroke%20Update.042009.pdf, p. 6) by affecting the coronary arteries that supply the myocardium with oxygen and nutrients. Peripheral arterial disease (“PAD”), which is an obstruction of the large arteries in the arms or legs, affects about 8 million Americans. (Id. at 21) Both of these conditions may be caused by the development and progression of atheromatous plaques in the vessel walls (atherosclerosis) or other inflammatory processes of the vessel wall.

Atherosclerosis is an inflammatory disease of the arterial vasculature. The lesions that characterize the disease result from an excessive inflammatory/fibroproliferative response to injury of endothelial cells that line the vessel wall. Endothelial injury may be caused by smoking, age, hypertension, diabetes, and hypercholesterolemia. Injury induces the endothelium to have procoagulant (instead of anticoagulant) properties and to release vasoactive molecules, cytokines and growth factors. These effects are responsible for the adherence, migration and accumulation of monocytes and T-lymphocytes, as well as for proliferation of underlying smooth muscle cells. The lesions progress through stages of varying complexity within the vessel wall, but are typically composed of activated T-lymphocytes, lipid-laden macrophages (foam cells), hyperproliferative smooth muscle cells, necrotic tissue, and adhered platelets, all enclosed in a fibrous cap. Macrophages, in particular, are present throughout the progression of atherosclerotic lesions. Local accumulation of macrophages may be enhanced by the angiogenic response of vasa vasorum that become much more permeable in proximity to the plaque.

Inflammation also plays a crucial role in inflammatory bowel disease. Inflammatory bowel disease is marked by abdominal pain, vomiting, diarrhea, rectal bleeding, severe internal cramps/muscle spasms in the region of the pelvis, weight loss and various associated complaints. Chronic inflammatory bowel disease consists of two main subtypes: Crohn's disease and ulcerative colitis (see, e.g., Podolsky, D., NEW ENGL. J. MED. 325:928 (1991)). The incidents of Crohn's disease continues to increase worldwide, reaching incidence rates ranging from 3.1 to 14.6/100,000 in North America and from 0.7 to 9.8/100,000 in Europe.

The main difference between Crohn's disease and ulcerative colitis is the location of the inflammation and nature of the tissue in which the inflammation occurs. Crohn's disease can be localized in any part of the gastrointestinal tract, although the most frequent location is the terminal ileum; observed in 90% of the patients with small-intestinal Crohn's disease, who in turn constitute 30-40% of all Crohn's disease patients. Crohn's disease is also known to affect the whole bowel wall. Ulcerative colitis, on the other hand, is typically restricted to the colon and rectum, with inflammation being limited to the mucosal lining of the gut.

Accurate diagnosis and assessment of inflammatory bowel disease are crucial for determining the proper course of treatment, including prophylactic regimens to reduce bouts of inflammation and surgical intervention.

### SUMMARY OF THE INVENTION

In a first embodiment of the invention, there is provided a method of obtaining a series of enhanced magnetic resonance images in a single imaging session with a subject. The method comprises administering a macrophage imaging agent to the subject during an administration session; allowing a passage of time sufficient for accumulation of the agent in macrophages of the subject; and in a single imaging session: (i) acquiring a macrophage-enhanced magnetic resonance image to target the macrophages; and (ii) acquiring a different magnetic resonance image targeting a physiological phenomenon other than macrophages. In particular embodiments, acquiring the different magnetic resonance image follows the acquisition of the macrophage-enhanced magnetic resonance image. Additional embodiments further comprise administering a contrast agent before acquiring the different magnetic resonance image. Yet other embodiments further comprise using the macrophage-enhanced magnetic resonance image to observe macrophage activity associated with a primary tumor or metastatic tumor. Additional embodiments further comprise using the macrophage-enhanced magnetic resonance image to observe macrophage activity associated with an atherosclerotic plaque. In particular embodiments, the macrophage imaging agent is an ultrasmall superparamagnetic iron oxide particle. In another particular embodiment, the macrophage imaging agent is ferumoxtran-10. In still another particular embodiment, the macrophage imaging agent is ferumoxytol. In still yet another embodiment, the ultrasmall superparamagnetic iron oxide particle comprises a carboxyalkylated reduced dextran iron oxide complex. In certain embodiments, sufficient passage of time for accumulation of the agent in macrophages is 24-144 hours. In particular embodiments, the different magnetic resonance image targets the vasculature or perfusion. In other embodiments, the physiological phenomenon is perfusion. In still other embodiments, the different magnetic resonance image targets the vasculature. In another particular embodiment, the contrast agent is selected from a group consisting of ultrasmall superparamagnetic iron oxide particles, superparmagnetic iron oxide particles, gadolinium compounds, manganese compounds, and perfluorocarbons. Other particular embodiments further comprise a third magnetic resonance image targeting a physiological phenomenon other than macrophages or perfusion. In certain embodiments, the physiological phenomenon other than macrophages or perfusion comprises the vasculature. In still another particular embodiment, the macrophage-enhanced magnetic resonance image comprises a whole-body MRI scan. In still yet another embodiment, the single imaging session provides information for identification, localization and characterization of inflammatory bowel disease. In another embodiment, the characterization of inflammatory bowel disease comprises assessing the efficacy of treatment.

In another embodiment of the invention, there is provided a method of assessing a stage of cancer in a single imaging session with a subject. The method comprises administering a macrophage enhancing contrast agent to the subject during an administration session; allowing passage of time sufficient for accumulation of the agent in macrophages of the subject; acquiring a first magnetic resonance image of regions of the subject's body at risk of cancer; using the image to assess macrophage density and displacement associated with any primary cancer or metastatic cancer in the subject, such density and displacement being indicative of neoplasia; and acquiring a second magnetic resonance image of the regions of the subject's body indicating neoplasia to measure a physiological phenomenon other than macrophages. In a particular embodiment, the macrophage enhancing contrast agent is ferumoxytol. In another particular embodiment, the passage of time is four days. In yet another particular embodiment, the cancer is breast cancer. In particular embodiments, the regions of the body comprise the breast and axillary, subclavian and sterna lymph nodes. In other particular embodiments, the second magnetic resonance image is acquired from liver and bone marrow. Yet other particular embodiments further comprise acquiring at least a third magnetic resonance image following administration of a second contrast agent to assess tumor angiogenesis. In certain embodiments, the second contrast agent is the same composition as the macrophage enhancing contrast agent. Still yet other particular embodiments further comprise acquiring a diffusion-weighted magnetic resonance image or performing magnetic resonance spectroscopy. Other particular embodiments further comprise restaging the cancer at a later time.

In another embodiment of the invention, there is provided a method of characterizing vulnerable atherosclerotic plaques in a single imaging session with a subject. The method comprises administering a macrophage imaging agent to the subject during an administration session; allowing passage of time sufficient for accumulation of the agent in macrophages of the subject; and in a single imaging session: (i) acquiring a first magnetic resonance image to observe active macrophage populations in a region of the subject's vasculature susceptible to atherosclerosis; and (ii) if an active macrophage population is observed, acquiring a second magnetic resonance image of the region to visualize a phenomenon other than macrophages. In particular embodiments, acquiring the second magnetic resonance image includes doing so by bright blood imaging or magnetic resonance angiography. Additional embodiments further comprise acquiring a high spatial resolution, steady state vascular-enhanced magnetic resonance image. In particular embodiments, the passage of time is 1-10 days.

In another embodiment, there is provided a method of assessing stage of cancer of a subject in a single imaging session. The method comprises administering a macrophage imaging agent to the subject during an administration session; allowing a passage of time sufficient for accumulation of the agent in macrophages of the subject; and in a single imaging session (i) acquiring a macrophage-enhanced magnetic resonance image of regions of the subject's body at cancer risk, wherein the image is used to stage any primary cancer or metastatic cancer in the subject by assessment of macrophage density, macrophage displacement or tumor morphology associated with the cancer; and (ii) acquiring a different magnetic resonance image targeting a physiological phenomenon other than macrophages. In a particular embodiment, using the image includes observing macrophage activity associated with a primary tumor or with any metastatic tumor in bone, lymph node, spleen, liver, central nervous system, lung, or other organ. In another embodiment, the regions collectively include the entire body. In yet another embodiment, the macrophage imaging agent is an ultrasmall superparamagnetic iron oxide particle. In still another particular embodiment, the macrophage imaging agent is a complex of ultrasmall superparamagnetic iron oxide and a polysaccharide. In yet another embodiment, the polysaccharide is selected from the group consisting of dextran, reduced dextran and a derivative thereof. Other embodiments further comprise administering a contrast agent before acquiring the different magnetic resonance image. In a particular embodiment, the contrast agent is gadolinium.

Still another embodiment of the invention provides a method of assessing efficacy of an anticancer treatment in a single imaging session with a subject. The method comprises administering a macrophage imaging agent to the subject during an administration session; allowing a passage of time sufficient for accumulation of the agent in macrophages of the subject; and in a single imaging session: (i) acquiring a first magnetic resonance image of at least one region of the subject's body to be targeted by an anti-cancer treatment to establish a pre-treatment image, such image configured to reveal an accumulation of macrophages resulting from a pathophysiological process associated with cancer, which would not be present in the absence of such a pathophysiological process; (ii) administering the anticancer treatment to the subject; and (iii) acquiring a second magnetic resonance image of the at least one region of the subject's body targeted by the anticancer treatment to establish a post-treatment image, such image configured to assess an extent of any reduction in the post-treatment image compared to the pre-treatment image with respect to tumor-associated macrophage density and displacement associated with a primary cancer or metastatic cancer in the at least one region, wherein the extent of reduction in tumor-associated macrophage density and displacement is indicative of the efficacy of the anti-cancer treatment. In a particular embodiment, the anticancer treatment includes a treatment selected from the group consisting of chemotherapy, extirpation, in situ ablation, radiation therapy, liposomal drug delivery, immunotherapy, gene therapy and alternative therapy. In another embodiment, the macrophage imaging agent is an ultrasmall superparamagnetic iron oxide particle. In yet another particular embodiment, the ultrasmall superparamagnetic iron oxide particle is a macrophage biomarker capable of being administered to a subject from between 12 and 168 hours prior to whole body MEMRI evaluation. In yet another embodiment, the at least one region is located outside of liver, spleen and lymph nodes of the subject.

In still another embodiment of the invention, there is provided a method of assessing inflammatory bowel disease in a single imaging session. The method comprises administering a macrophage imaging agent to the subject during an administration session; allowing a passage of time sufficient for accumulation of the agent in macrophages of the subject; and in a single imaging session (i) acquiring a macrophage-enhanced magnetic resonance image of regions of the subject's abdomen, wherein the image is used to assess macrophage density potentially associated with a region of inflammatory bowel disease; and (ii) acquiring a different magnetic resonance image of the potential region of inflammatory bowel disease targeting a physiological phenomenon other than macrophages.

### BRIEF DESCRIPTION OF THE DRAWINGS

The foregoing features of the invention will be more readily understood by reference to the following detailed description, taken with reference to the accompanying drawings, in which:

FIG. 1 shows an illustration of a patient placed within a whole-body MRI system for scanning, here used with a currently-approved contrast agents to visualize the arterial vessels throughout the body. (see Nael et al. (2007) Am. J. Radiol , 188, 529-39).

FIGS. 2A-C show three variations of a cancer report card that may be used when practicing embodiments of the present invention.

FIG. 3 is an illustration showing a tumor region with increased macrophage density, and the process whereby tumor-associated macrophages produce chemotactic factors (CC-Chemokines, e.g. CCL2), macrophage colony stimulatory factor (M-CSF) and vascular endothelial growth factor (VEGF) to generate new blood vessels and facilitate further growth of the tumor (angiogenesis) (illustration from Allavena et al., (2006) Eur. J. Cancer, 42, 717-727).

FIGS. 4A and 4B show a patient with breast cancer with macrophages around the primary tumor and displaced from the metastatic tumor in an adjacent lymph node tumor. FIG. 4A is an in vivo MRI of the patient's breast with a contrast agent of the invention; FIG. 4B is an in vitro MRI of the removed specimen containing the tumor and a metastatic lymph node, also with a contrast agent of the invention. The arrow in FIG. 4A shows the very clear presence of a dark accumulation of macrophages indicating a tumor. FIG. 4B shows the lymph node tumor indicated by a dark outline of macrophages where the center area of the tumor is light, because the cancer cells have displaced the macrophages from this central region of the tumor.

FIGS. 5A and 5B show a patient with bladder cancer with macrophages around the primary tumors. The bladder is indicated generally with an arrow in FIGS. 5A and 5B as the large central light area in the center of the pelvis region. FIG. 5A is the MRI without contrast agent and FIG. 5B is the image with contrast agent of the invention. In FIG. 5A, the tumor's presence is only hinted at by the “crease” in the bladder (shown with an arrow) that seems to be an indication of pressure on or displacement of the bladder along this juncture. FIG. 5B, with contrast agent, clearly shows the line of demarcation for the tumor along that “crease”, with the massive tumor showing as a dark mass directly to the left of this line, continuing down to a point and back again. A second smaller tumor is indicated with an arrow to the right of the bladder, appearing as a bulls eye type node. This second tumor is outlined with a dark ring of macrophages. The center of the tumor shows up lighter where the cancer cells have displaced the macrophages. As an indication of the power of this contrast agent in cancer diagnosis and staging, this tumor is not identifiable as a tumor at all in the image (5A) with no contrast agent.

FIGS. 6A, 6B and 6C show MRI depictions of a patient with prostate cancer. The prostate is indicated generally as the large central circular space in the center of the pelvis region. FIG. 6A is the MRI without contrast agent, and FIGS. 6B and 6C are the MRIs with a contrast agent of the invention. The presence of a tumor is not indicated at all in FIG. 6A, the MRI without contrast agent. In stark comparison, FIGS. 6B and 6C indicate the presence of a very large tumor, and possibly multiple large tumors, within the prostate, as indicated by the three arrows pointing out regions of the tumor (or tumors) that are particularly enhanced with macrophage, in the presence of contrast agent. In FIG. 6C, one can more clearly see the large size of the tumor, as well as its amorphous nature (indicated by an arrow to the central left portion of the prostate), where macrophage have infiltrated the tumor and cause the tumor to appear mottled dark and light grey in this image. The presence of the macrophages provides important information about the aggressive nature of this prostate cancer.

FIG. 7 is a time- and tissue-dependent graph of the in vivo distribution of a macrophage imaging agent used in embodiments the invention.

FIG. 8 is a flow diagram illustrating the steps of a MRI protocol used to image potential macrophage location prior to the administration of a macrophage-seeking contrast agent, followed by subsequent imaging after sufficient accumulation of this biomarker in the macrophage target.

FIG. 9 is a flow diagram illustrating the steps of the composite MEMRI protocol for imaging macrophages after accumulation of a macrophage-seeking biomarker contrast agent, according to an embodiment of the invention.

### DETAILED DESCRIPTION OF SPECIFIC EMBODIMENTS

Definitions. As used in this description and the accompanying claims, the following terms shall have the meanings indicated, unless the context otherwise requires:

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, melanoma and various types of head and neck cancer.

“Tumor” and “neoplasm” as used herein refer to any mass of tissue that result from excessive cell growth or proliferation, either benign (noncancerous) or malignant (cancerous) including pre-cancerous lesions.

“Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

As used herein, the terms “atherosclerosis,” “plaque” or “lesion” refer to a hardening and/or increase in the size and number of cells and underlying smooth muscles cells of a given area of artery. The atherosclerotic area, plaque or lesion may be comprised of (but not limited to) endothelial cells, activated T-lymphocytes, macrophages, smooth muscle cells of a given arterial wall, necrotic tissue, and platelets, and may be enclosed in a fibrous cap.

As used herein, the term “subject” refers to any animal (e.g., a mammal), including, but not limited to humans, non-human primates, rodents, and the like, which is to be the recipient of a particular treatment. Typically, the terms “subject” and “patient” may be used interchangeably herein in reference to a human subject.

The terms “cancer cell”, “tumor cell” and grammatical equivalents refer to the total population of cells derived from a tumor including both non-tumorigenic cells, which comprise the bulk of the tumor cell population, and tumorigenic cells.

As used herein, “ assessing stage of cancer ” or “staging cancer” refers to any MRI information that is useful in determining whether a patient has a primary cancer or tumor, and/or metastatic cancer or tumor, and/or information that is useful in classifying the stage of the cancer into a phenotypic category or any category having significance with regards to the prognosis of or likely response to anticancer treatment (either anticancer treatment in general or any particular anticancer treatment) of the primary or metastatic tumor(s). Similarly, assessing stage of cancer refers to providing any type of information, including, but not limited to, whether a subject is likely to have a condition (such as a tumor), and information related to the nature or classification of a tumor as for example a high risk tumor or a low risk tumor, information related to prognosis and/or information useful in selecting an appropriate treatment. Selection of treatment can include the choice of a particular chemotherapeutic agent or other treatment modality such as surgery or radiation or a choice about whether to withhold or deliver therapy.

As used herein, the terms “providing a prognosis”, “prognostic information”, or “predictive information” refer to providing information regarding the health of a subject or the impact of a disease and/or disease treatments on said subject. For example, the terms may refer to providing information regarding the impact of the presence of cancer (e.g., as determined by the staging methods of the present invention) on a subject's future health (e.g., expected morbidity or mortality, the likelihood of getting cancer, and the risk of metastasis).

As used herein, the term “characterize a vulnerable plaque,” and grammatical equivalents thereof, refers to providing information regarding the cellular content, size, location, morphology and histology of an atherosclerotic plaque or lesion.

The term “imaging protocol” refers to the series of steps and settings necessary to acquire a magnetic resonance image, including subject visits, administration of contrast agents, waiting period, imaging session(s) and scanner settings.

The term “imaging session” refers to acquiring one or more MR images of a subject at a single location within a given period of time. For example, an “imaging session” may refer to a visit by a subject at an MRI imaging facility on an occasion wherein three separate MRI scans are performed on the subject over the course of several hours. The scanning time may be continuous or divided into discrete sub-sessions.

The term “perfusion-enhanced MRI” (“PEMRI”) refers to any MRI technique or protocol that produces an image or measurement related to regional blood flow in tissues. The PEMRI is performed by “tagging” blood during image acquisition. Tagging may be achieved by bolus injection of a contrast agent or by saturating the protons in aterial blood with RF inversion or saturation pulses. PEMRI may require fast or ultra-fast imaging methods before, during and/or after bolus injection of the contrast agent. Other PEMRI techniques utilized in embodiments of the invention may use a bolus injection of a contrast agent during ultra-fast T2 or T2* acquisitions. PEMRI may be used to evaluate conditions associated with, but not limited to, cancer, vascular disease, ischemic disease, metabolism, malignancy, metastases, stroke, heart disease and kidney disease.

The term “vascular-enhanced MRI” (“VEMRI”) refers to any MRI technique or protocol that may be used to evaluate the cardiovascular system. These techniques include, for example, MR images acquired with imaging options known to those of skill in the art to enable vascular visualization, such as those disclosed in U.S. Pat. No. 5,314,679 incorporated by reference herein, and magnetic resonance angiography (“MRA”) to visualize moving blood, with or without the use of contrast agents.

The term “composite MEMRI protocol” refers to use of a MEMRI technique in conjunction with other specialized MRI techniques, including, for example, PEMRI and VEMRI.

Embodiments of the present invention include macrophage-enhanced MRI (“MEMRI”) imaging protocols capable of imaging macrophages in areas of the body where their presence indicates a pathophysiological process or disease state. For example, embodiments of the present invention utilize macrophage-enhancing contrast agents within tumor-associated macrophages (“TAMs”) to provide contrast around, and hence visualization of, primary or metastatic tumors, thereby permitting cancer staging and assessment of the efficacy of an anticancer treatment. Specific embodiments of the present invention comprise administering a macrophage imaging agent to a subject, making a magnetic resonance image of regions of the subject's body at risk for or afflicted by cancer, and using the image to assess macrophage density or displacement associated with the primary or metastatic tumor, thereby assessing the presence or stage of cancer, or the effectiveness of an anti-cancer treatment. Embodiments of the present invention are also applicable in imaging vascular diseases such as atherosclerosis and chronic inflammatory diseases such as inflammatory bowel disease.

Embodiments of the present invention enable performing MEMRI as a component of a composite MRI protocol completed in a single imaging session. In particular, it has been surprisingly found that proper use and selection of an ultrasmall superparamagnetic iron oxide particle (USPIO) as an MRI contrast agent in a MEMRI can eliminate the need to conduct MRI scans on separate occasions before and after administration of the contrast agent. Instead, the macrophage imaging agent can be administered during a relatively brief administration session followed by the passage of a sufficient amount of time to allow the macrophage imaging agent to accumulate in macrophages. In various embodiments, after administration of the contrast agent, the subject can go home for one to three days and then return for an imaging session. The appropriate delay between injection and imaging allows the MR agent to be selectively distributed in macrophages where the effect upon MR signal with an appropriate pulse sequences is sufficiently distinctive to eliminate the need for pre-contrast scans while, surprisingly, maintaining the superior diagnostic capabilities of MRI. The images produced by the MEMRI scan may then be correlated with a potential disease state (e.g., atherosclerosis) and a second MRI performed at the same imaging session to provide additional targeted physiological information on that disease state, thereby completing the composite MRI protocol.

The additional MR imaging may by accompanied by the injection of an MR contrast agent that enhances other important tissues in relatively short times. The additional agent may be selected from gadolinium agents such as small chelates like Magnevist®, blood pool agents like Ablavar®, or biliary agents like Eovist®. The additional agent may also be a USPIO such as ferumoxytol, which has an early enhancement effect upon blood, producing information about perfusion, regional blood volume, or vascular anatomy.

Magnetic Resonance Imaging (“MRI”) is noninvasive, tomographic, nonionizing, and non-invasive imaging modality able to generate images with high resolution and excellent soft tissue contrast. It is also able to generate high-contrast images of the interior of the human body and provides diagnostic information on selected body regions. By taking advantage of inherent tissue differences in MR properties that can be imaged by varying the MR image sequences, MRI has proven useful in the diagnosis of many diseases such as, cancer, multiple sclerosis, sports related injury, vascular disease and bone marrow disorders. MRI provides unique imaging capabilities that are not attainable in any other imaging methods. For example, MRI can provide detailed images of soft tissues, abnormal tissues such as tumors, and other structures which cannot be readily imaged using techniques like X-rays. Further, MRI operates without exposing patients to ionizing radiation that is integral to x-ray or scintigraphic imaging.

MRI involves the interrogation of the nuclear magnetic moments of a subject placed in a strong magnetic field with radio frequency (RF) pulses. An MRI system typically comprises a fixed magnet to create the main strong magnetic field, a gradient coil assembly to permit spatial encoding of signal information, a variety of RF resonators, which are herein called “RF coils”, to transmit RF energy to, and receive signals emanating back from the subject being imaged, and a computer to control overall MRI system operation and create images from the signal information obtained. The large majority of RF coils used in MR imaging are tuned to ¹H due to the high abundance of this paramagnetic nucleus in the body, although MRI using other nuclei (¹³C, ³¹P, ²³Na, ¹⁹F) are also possible.

MR active nuclei that have a net charge and motion automatically acquire a magnetic moment and can align with an externally applied magnetic field. For example, a hydrogen nucleus with a magnetic moment acts as a small magnet, and some of the nuclei will align parallel to the external magnetic field while others align in an anti-parallel. Each hydrogen nucleus is also spinning on its axis, which under the influence of the magnetic field, gains a unique precessional frequency. When exposed to pulses of electromagnetic energy (e.g., radio waves) close to their precessional frequency, the MR active nuclei resonate. When an RF pulse is applied, the energy is absorbed by the protons. The absorption of energy temporarily causes the protons to move out of alignment with the magnetic field. This effect causes the net magnetization vector (“NMV”) of the nuclei to flip to a transverse plane that is aligned at 90° with respect to the magnetic field. Another result of resonance is that the magnetic moments of the individual nuclei move to the same position on their precessional paths and are said to be “in phase”. Therefore, as a result of resonance, in phase magnetization occurs with precession at a precise frequency in the transverse plane. By Faraday's law, this moving magnetic field can induce voltage in a receiving coil, which generates an MR signal.

The major hardware that comprises an MRI system includes the magnet, cryogenic systems, gradient coils, RF coils, patient table, the various amplifiers and image acquisition and processing subsystems. A whole bodywhole-body scanner typically requires a large enough magnet opening to accommodate whole bodywhole-body scans with sufficient magnetic field homogeneity, RF field homogeneity and enough RF power over large volumes to generate sufficient excitation, sufficient gradient linearity over a large volume, strength and slew rate to generate images of acceptable clarity and quality to make diagnosis of diseased organs and tissues. These in turn depend on the magnetic field strength and patient opening which determine to a large extent the overall system design, power consumption and demand on the complexity of the electronics and image acquisition and processing systems.

When the RF pulse is removed, the NMV realigns with the magnetic field as the individual nuclei lose energy, a process called relaxation. At the same time, the magnetic moments of the hydrogen dephase. As the NVM returns to match the external magnetic field, the amount of magnetization generated by the nuclei in a plane aligned with the main magnetic field gradually increases, in a process called “recovery”. Correspondingly, the magnetization in the transverse plane decays. The recovery of longitudinal magnetization is caused by processes known as T1 recovery, while the decay of transverse magnetization is termed T2 decay. The rate of recovery and decay are referred to as T1 and T2 relaxation times, respectively. Various sequences and durations of RF pulses, signals and periods of recovery can be manipulated to produce contrast in MRI images. Exemplary pulse sequence types include spin echo, inversion recovery and gradient echo. The pulse sequences can be controlled to reflect the T1 and T2 relaxation rates, thereby favoring or weighting one over the other and altering the contrast between fat and water. For example, T1 weighted images are characterized by bright fat and dark water. T2 weighted images produce bright water and dark fat. T2 processes are useful in imaging of organs such as the liver, spleen, or lymph nodes that contain lesions such as tumors.

Generally, the differences related to relaxation time constants T1 and T2 of water protons in different environments are used to generate an image. However, these differences can be insufficient to provide sharp high resolution images with adequate depiction of health or disease. Contrast agents increase the diagnostic capabilities of MRI by affecting the normal relaxation times, principally on the protons of water, thereby allowing the differentiation of diseased and normal tissue. All contrast agents have both T1 and T2 properties; however, either T1 or T2 relaxation can characterize the dominant relaxation property of a particular contrast agent. An additional relaxation property is T2*, which is the spin-spin relaxation time as influenced by variations of molecular interactions and intrinsic magnetic heterogeneities of tissues in the magnetic field.

Examples of contrast agents include a number of magnetic agents paramagnetic agents (which primarily alter T1) and ferromagnetic or superparamagnetic (which disproportionately alter T2 response). Chelates (e.g., EDTA, DTPA and NTA chelates) can be used to attach (and reduce toxicity) of some paramagnetic substances (e.g., Fe⁺³, Mn⁺², Gd⁺³). Other agents can be in the form of particles, e.g., less than 10 μm to about 10 nM in diameter). Particles can have ferromagnetic, antiferromagnetic or superparamagnetic properties. Particles can include, e.g., magnetite (Fe₃O₄), gamma-Fe₂O₃, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like.

Positive contrast agents cause a reduction in the T1 relaxation time (increased signal intensity on T1 weighted images). They are typically small molecular weight compounds containing as their active element Gadolinium, Manganese, or Iron. All of these elements have unpaired electron spins in their outer shells and long relaxivities.

The most popular agents for assessing the primary tumor are small gadolinium chelates. Given intravenously, these agents are distributed by blood perfusion and can identify regions of excess vascular leakiness or enlarged extracellular spaces that may herald the presence of cancer. However, these agents have no cell targeting capabilities and their distribution and accumulation are not specific for cancer nor for the tissue at risk for cancer metastasis. Bolus administration and dynamic MRI may provide some additional information about the degree of vascular leakiness, but such information can only be obtained for one body region of interest. As is described in example 1 (vide infra), the use of such contrast-enhanced MRI may be insufficient to characterize the cancer stage even with respect to the primary tumor.

Negative contrast agents (appearing predominantly dark on MRI) are small particulate aggregates often termed superparamagnetic iron oxides (SPIOs) or ultrasmall superparamagnetic iron oxides (USPIOs). USPIOs typically are less than about 100 nanometers in diameter, and often have a mean diameter of less than 50 nm. These agents produce predominantly spin-spin relaxation effects (local field inhomogeneities), which results in shorter T1 and T2 relaxation times. Generally, the size of a contrast agent is inversely proportional to its duration in the vascular system, and the small size of these molecules permits a long blood half-life. These agents produce predominantly spin-spin relaxation effects (local field inhomogeneities), which result in shorter T1 and T2 relaxation times, depending on concentration. SPIOs and USPIOs usually consist of a crystalline iron oxide core containing thousands of iron atoms and a shell of a polymer, such as dextran or polyethyleneglycol, and can produce very high T2 and T2* relaxation times when accumulated in a tissue of interest, which produces a focal signal void. Ferumoxytol and ferumoxtran-10 are examples of USPIO contrast agents. In one study, useful iron oxide nanoparticles such as ferumoxtran-10 were studied for their effect on macrophages in vitro and found to be non-toxic to human monocyte-macrophages (see Gillard et al., Biomaterials 28 (2007) 1629-1642).

The coatings surrounding the USPIO iron core have a profound influence on in vitro stability and in vivo distribution and duration. The type of polymer or other molecule utilized imparts different physiochemical properties (e.g., size, electrical surface charge and density of covering) to each USPIO. The most common coatings are polymers such as dextran, carboxymethylated dextran, carboxy dextran, polyethyleneglycol or other derivatives thereof. Notably, there is no precise structure-activity relationship that allows accurate prediction of stability, biodistribution, clearance from the vascular system and safety. DiMarco, M. et al., Physiochemical characterization of ultrasmall superparamagnetic iron oxide particles (USPIO) for biomedical application as MRI contrast agents, INTL. J. NANOMED., 2: 609-622, 610 (2007).

A special group of negative contrast agents (appearing dark on MRI) are perfluorocarbons (perfluorochemicals), because their presence excludes the hydrogen atoms responsible for the signal in MR imaging. These agents are reported to allow enhanced, sensitive detection and quantification of occult microthrombi within the intimal surface of atherosclerotic vessels in symptomatic patients and provide direct evidence to support acute therapeutic intervention, particularly if used in combination with gadolinium (see Flacke et al. CIRCULATION. 2001; 104: 1280).

Whole-body (or full-body) MRI technology has been known and used for a number years. For example, U.S. Pat. No. 6,963,768 to V. B. Ho and T. K. F. Foo issued Nov. 8, 2005 (Whole-body MRI scanning with moving table and interactive control), U.S. Pat. No. 6,681,132 issued Jan. 20, 2004 to J. Katz et al. (Sodium magnetic reasonance imaging used in diagnosing tumors and assessing response to treatment), U.S. Pat. No. 6,975,113 issued on Dec. 13, 2006 to D. Gun (Method and system for moving table MRI with partial Fourier imaging) and U.S. Pat. No. 7,227,359 issued Jun. 5, 2007 to J. Ma (Method and apparatus for phase-sensitive magnetic resonance imaging) all describe various methods and systems that can be used for performing continuous whole-body MRI. Similarly, U.S. Publication No. 2005/0171423 to V. B. Ho and T. K. F. Foo published Aug. 4, 2005 (Whole-body MRI scanning with moving table and interactive control) and U.S. Publication No. 2005/0154291 to L. Zhao et al. published Jul. 14, 2005 (Method of using a small MRI scanner) also disclose whole-body MRI methods and apparatus. It is envisioned that any one or more of the above-disclosed methodologies and apparatus may be useful to carry out various embodiments of the presently claimed invention. With that in mind, the entire contents of the above-referenced U.S. Pat. Nos. (6,963,786; 6,681,132; 6,975,113; 7,227,359) and U.S. Published Applications (2005/0171423 and 2005/0154291) are hereby incorporated by reference herein in their entirety.

Whole-body MRI has recently been used for evaluation of metastasis in bone in the absence of contrast agents. When MRI has been used in tumor staging, it has been by taking advantage of inherent tissue differences in MR properties that could be imaged by varying the MR image sequences. To obtain whatever information is contained in these inherent tissue differences for all tissues at cancer risk has required selection of different imaging sequences for each potential host tissue as well as the repeated programming and positioning of the patient within the MR instrument.

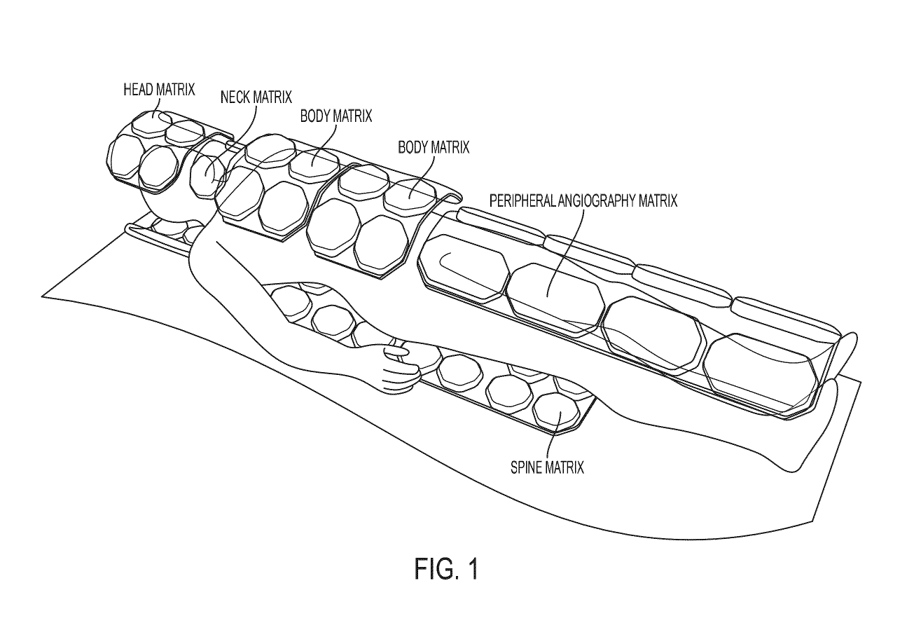

Traditional magnet systems for MRI scanners have to accommodate the insertion of a human being and generate a homogeneous region large enough to cover a cylindrical area with a diameter between about 20 to about 50 cm, preferably about 40 cm, spherical volume (DSV) over the subject. For sufficient image quality, the magnets are typically made from permanent magnets in low-field systems (<5,000 gauss; <0.5 T) and superconducting magnet systems in high field systems (>10,000 gauss; >IT). FIG. 1 shows an illustration of a patient placed within a whole-body MRI system for scanning with the use of contrast agents (see Nael et al. (2007) AM. J. RADIOL, 188, 529-39).

Embodiments of the present invention include methods for staging, diagnosing, characterizing, and assessing cancer progression, growth and potential for and/or actual metastasis using MRI and a contrast agent. Some MRI contrast agents that may be useful in carrying out the presently claimed invention are summarized in EP0502814B1, the contents of which are hereby incorporated by reference herein.

For all cancers, staging requires information on the status of the primary tumor, the regional lymph nodes, and the evaluation of possible metastatic sites. At each of these locations, usually evaluated using the TNM system as described for breast cancer above, the activity of local macrophages provides diagnostic information. In primary tumors or metastatic sites, increased macrophage density identifies a local region of concern. In addition, the displacement of normal macrophages from lymph nodes, liver, or spleen, when appropriate to the primary tumor, identifies potential metastasis. In particular embodiments of the present invention methods of staging cancer involves whole-body MRI using a macrophage-seeking contrast agent.

With the feasibility of whole-body MRI scanning and the availability of an MR biomarker that accumulates in local macrophages, it becomes feasible to conduct whole-body TNM staging in a single examination. One particularly useful class of MR biomarkers providing this utility are iron oxide nanoparticles. An important attribute facilitating their utility is a long blood life so that better macrophage accumulation is achieved. Two such agents are ferumoxytol and ferumoxtran-10, contrast agents that are particularly suited for use in embodiments of the presently claimed invention. Ferumoxytol and ferumoxtran-10 are MRI agents that are superparamagnetic and USPIOs. In general, USPIOs that comprise polyols, polyethers and/or polysaccharides, particularly reduced polysaccharides, more particularly carboxyalkylated reduced polysaccharides are useful for embodiments of the whole-body MRI scanning described here. In a particular embodiment, the polysaccharide of the USPIO is a carboxyalkylated reduced dextran iron oxide complex.

After administration, contrast agents of the invention such as ferumoxtran-10 and ferumoxytol slowly escape from the blood vessels over the course of 12 to 168 hours (or potentially longer) by leaking into the interstitial space. There, they encounter monocytes that have been recruited through cytokine signals to the tumor, atherosclerotic lesion or inflamed tissue and have been differentiated into macrophages. The macrophages will internalize (phagocytose) the compound, enabling imaging of the macrophages, whether they are tumor-associated or plaque-associated. In this manner, ferumoxtran-10 and ferumoxytol function as particularly useful macrophage imaging agents. For example, ferumoxtran-10 has been used in the diagnosis of atherosclerosis both in animal and human studies. See, e.g., Hyafil, F. et al., Ferumoxtran-10-Enhanced MRI of the Hypercholesterolemic Rabbit Aorta, ATERIOSCLER THROMB VASC BIOL. 26:176-181 (2006); Trivedi, R. A. et al., Identifying Inflamed Carotid Plaques Using In Vivo USPIO-Enhanced MR Imaging to Label Plaque Macrophages, ATERIOSCLER THROMB VASC BIOL. 26: 1601-1606 (2006).

More particularly, MRI agents useful for embodiments of the presently claimed invention will be macrophage-seeking agents, such as the USPIOs disclosed in the following patents and applications, the contents of which are all hereby incorporated by reference herein in their entirety: U.S. Pat. No. 5,160,726 issued Nov. 3, 1992 to Josephson et al. (Filter Sterilization for Production of Colloidal Superparamagnetic MR Contrast Agents); U.S. Pat. No. 5,262,176 issued Nov. 16, 1993 to Palmacci et al. (Synthesis of Polysaccharide Covered Superparamagnetic Oxide Colloids); U.S. Pat. No. 6,599,498 issued on Jul. 29, 2003 to Groman et al. (Heat Stable Colloidal Iron Oxides Coated With Reduced Carbohydrates and Carbohydrate Derivatives); and U.S. Publication No. 2003/0225033 A1, published Dec. 4, 2003 to Groman et al. (Heat Stable Colloidal Iron Oxides Coated With Reduced Carbohydrates and Carbohydrate Derivatives); and U.S. Publication No. 2003/0232084 A1, published Dec. 18, 2003 to Groman et al. (Polyol and Polyether Iron Oxides Complexes Coated With Reduced Carbohydrates and Carbohydrate Derivatives). In particular embodiments, the contrast agent is used as a single contrast agent. In related embodiments, the contrast agent is used in combination with another contrast agent.

Traditionally, MRI is used to evaluate tumor morphology at a single site. For example, Combidex, a monocrystalline iron oxide complex useful for practicing the present invention, has been used experimentally to evaluate metastasis to lymph nodes—visualizing the displacement of the rich macrophage population in normal nodes (see Weissleder et al., N. ENGL. J. MED., 2003).

It has been surprisingly determined that administration of any one of a class of macrophage-seeking contrast agents followed by a whole-body MRI enables visualization of tissue surrounded by or associated with macrophages, which tissue will be enhanced in the MR image by the macrophage-seeking contrast agent. This in turn permits staging of any solid tumor, with the identification of both primary and metastatic cancers. In addition, such MRI methods allow an assessment of anticancer therapy, by comparison of tumor number, size, morphology and location, among other characteristics, observed with MRI before treatment, between treatment cycles and after the anticancer treatment.

Using macrophage-seeking contrast agents and whole-body MRI to perform a MEMRI evaluation as described above unexpectedly and surprisingly allows a physician to efficiently stage cancer for a variety of tumor types as well as assess metastasis at a much earlier point in the patient's cancer management because any tissue or organ in the entire body that has become surrounded by or associated with macrophages—a marker of the tumorigenic capabilities of that tumor—will be visualized by the whole-body MEMRI performed with any of the macrophage-seeking contrast agents described in particular embodiments of the present invention. By taking advantage of this effect in embodiments of the present invention, the physician can (a) provide a more accurate assessment of the metastatic potential of the primary tumor, (b) determine the degree of metastasis that may have already begun, (c) identify the location of the metastatic tumors, (d) customize the anticancer treatment based on the characteristics and metastatic extent of the primary tumor (or metastatic tumors already present), and (e) assess the efficacy of such treatment.

Recently it has become known by those specializing in MRI that whole-body imaging is becoming more feasible and will be useful in oncology, including staging. Thus, in addition to the patents for whole-body MRI described above, Paula Gould, in an “Overread” article in “Diagnostic Imaging” magazine discusses how whole-body MR imaging “should now be regarded as the test of choice for staging skeletal metastatic disease” (“Whole-Body MR Imaging Outclasses Bone Scans in Diagnostic Imaging”, Apr. 1, 2007).

However, the whole-body MR imaging advocated for staging skeletal metastatic disease does not propose using macrophage-seeking contrast agents to perform a MEMRI, and more importantly, misses the reason it would be advantageous to do so, not just for skeletal changes, but for the unexpected presence of macrophages. In fact, the article continues to stress that positron emission topography (PET)/computer tomography (CT), i.e. PET/CT, “is currently the best option for staging soft-tissue metastatic disease.” And, although acknowledging that whole-body MRI (again, in the absence of macrophage-enhancing contrast agents) is showing promise, a noted professor of musculoskeletal radiology in Dublin is quoted as stating that, while the emergence of diffusion-weighted techniques with whole-body MRI produce a PET-like map of the molecular movements of water, “sclerotic metastases do not have increased diffusion and will be missed using this technique.” And in April 2007, Quon et al. (RADIOLOGY, 243, pp. 204-211) continue to advocate the use of integrated FDG PET/CT imaging for detection, monitoring and its positive predictive value (PPV) for patients with bone metastases, mentioning only in the last sentence that “an additional adjunctive examination (e.g. MR imaging or biopsy) may be necessary” for patients with solitary bone lesions with discordant PET and CT findings. As with other reports, the authors do not suggest MR imaging with contrast agents, and in this case, do not suggest whole-body imaging, and never disclose use of macrophage biomarkers to perform a MEMRI evaluation.

Also, Ruehm et al. (JAMA, 2003, 290, pp 3199-3206) compared the strengths of whole-body fluorine 18 fluorodeoxyglucose (FDG) PET/CT and whole-body MRI for tumor staging in oncology (for a variety of malignant diseases), but do not disclose or suggest using MR contrast agents, particularly macrophage-seeking contrast agents, to stage cancer. Moreover, the authors concluded that “[r]eflecting the more precise definition of the T-stage and N-stage status, staging of malignancies was considerably more accurate when based on whole-body PET/CT imaging compared with whole-body MRI. Based on our data, FDG-PET/CT can be recommended as a first-line modality for whole-body tumor staging.” (see Ruehm et al, p. 3204, col. 1).

Nixon et al. compared MR imaging in patients with malignant brain tumors using iron oxide nanoparticles versus gadolinium agents as the contrast agents, concluding that the iron oxide contrast agent appears to enhance areas that do not enhance with gadolinium agents and may improve post-operative imaging problems associated with gadolinium. This study focused on primary tumors in the brain, and the authors report that there was a pattern of sharply delimited cells without processes that were histologically identifiable as macrophages and another pattern of stellate-shaped cells typical of reactive astrocytes, leading them to conclude that uptake in primary brain tumors of the iron oxide contrast agent studied is primarily concentrated in reactive cells in and around the brain tumor, rather than the tumor cells themselves and so could not conclude that all of the lesions imaged with the iron oxide contrast agent were actually tumors. The authors also hypothesized that changes in residual post-operative enhancement by the iron oxide contrast agent in brain lesions compared with what is observed with gadolinium contrast agents may be caused by trauma from surgery. Nothing in the study suggests that the iron oxide nanoparticles could be useful for whole-body imagining and staging of cancer in general using MEMRI evaluation.

A study in the New England Journal of Medicine by Weissleder et al. (N. ENGL. J. MED. 2003, 348, pp. 2491-2499) discloses the use of MRI for detection of clinically occult lymph node metastases in prostate cancer, reporting that MRI “is relatively insensitive for the detection of lymph-node metastases [but] can be improved by using different imaging agents and acquisition techniques” particularly the use of lymphotrophic superparamagnetic nanoparticles. The authors report 100% sensitivity in identifying patients with metastases using this technique, and 96% accuracy in correctly diagnosing patients that are free of lymph node metastases (see Weissleder at 2495). However, the technique is described for detecting lymph node metastases only, and nowhere do the authors suggest that this technique is generally applicable to other metastatic diseases. A follow-up study published in 2006 by Siemens Medical Solutions USA, Inc. (Harisinghani et al., 09 2006, SIEMENS MEDICAL SOLUTIONS USA INC., Order No. A9119-61365-C1-4A00, MR lymphangiography—Molecular Imaging Perspective with MR) confirmed the value of MR lymphangiography using lymphotrophic superparamagnetic nanoparticles for detecting/identifying lymph node metastases, but again did not suggest the techniques as generally applicable to other metastatic diseases other than lymph node metastases.

It has also become known by those in the area of cancer that macrophages are closely associated with tumor cells and are associated with metastasis. For example, Allavena et al., in a paper about tumor-associated macrophages as potential targets of anticancer therapy, discuss that “accumulation of leukocyte subpopulations is the hallmark of several pathological conditions, including tumors, and that a major component of the leukocytes found in tumors is macrophages (EUR. J. CANCER, (2006), 42, pp. 717-727 at 717).

Embodiments of the present invention, however, utilize the insight that the macrophage-seeking properties of certain MR contrast agents can be combined with whole-body MR imaging and surprisingly permit initial staging of a wide variety of soft tissue cancers, identification of primary and metastatic tumors with MRI using a single contrast agent, permit assessment of anticancer therapy and development of individualized therapy based on the morphology of the tumors identified, identify a site for biopsy, and provide a prognosis, because of the knowledge that macrophages associate with tumors and are an indicator of poor prognosis.

Embodiments of the present invention incorporate the surprising benefit that can be obtained by performing whole-body MEMRI to stage soft tissue cancers, allowing earlier, more sensitive, and more accurate evaluation of a wide variety of metastatic tumors using an MR contrast agent that accumulates in macrophages. None of the studies summarized above realized the potential for whole-body MEMRI in cancer diagnosis, staging, anticancer therapy, biopsy, prognosis, and follow-up therapy. Until this discovery, it was not understood that certain contrast agents, such as the lymphotrophic iron oxide nanoparticles disclosed in Weissleder at al. and the Siemens Medical Solutions USA Inc. report had all the properties required for such improved cancer evaluation using MEMRI. The prior art teaches particular contrast agents for particular tissue imaging, whole-body imaging in the absence of contrast agents to stage bone cancer, MRI with USPIOs to assess lymph nodes for cancer metastasis, and compares gadolinium contrast agents with USPIOs in brain cancer with MRI, but no where does the prior art suggest or teach that a general, non-tumor, but macrophage-seeking contrast agent with a long half-life might be used effectively with whole-body MEMRI for staging, diagnosing, assessing, providing prognosis, (and more) of soft tissue primary and metastatic tumors. In fact, a study by Guerbet of France using a USPIO contrast agent—Sineram, also known as Combidex in the U.S.A.—teaches away from Applicants surprising insight. The Guerbet study reported that characterization of breast tumors using MRI after administration of the contrast agent Sineram was not useful because no enhancement shortly after Sineram administration was seen in any of the assessed breast tumors by MRI, but all were detected using a gadolinium contrast agent (unpublished study, attached as Appendix A).

However, embodiments of the present invention show that the disclosed contrast agents, which have been used primarily to image macrophage displacement in lymph node, liver, spleen, can be exploited because of their general, non-tumor-specific macrophage seeking properties and long in vivo half life, to be used with whole-body MEMRI to identify macrophage enriched regions associated with cancer foci, thereby enabling the physician to stage cancer, follow metastasis, assess prognosis, and assess anticancer treatments, among other benefits.

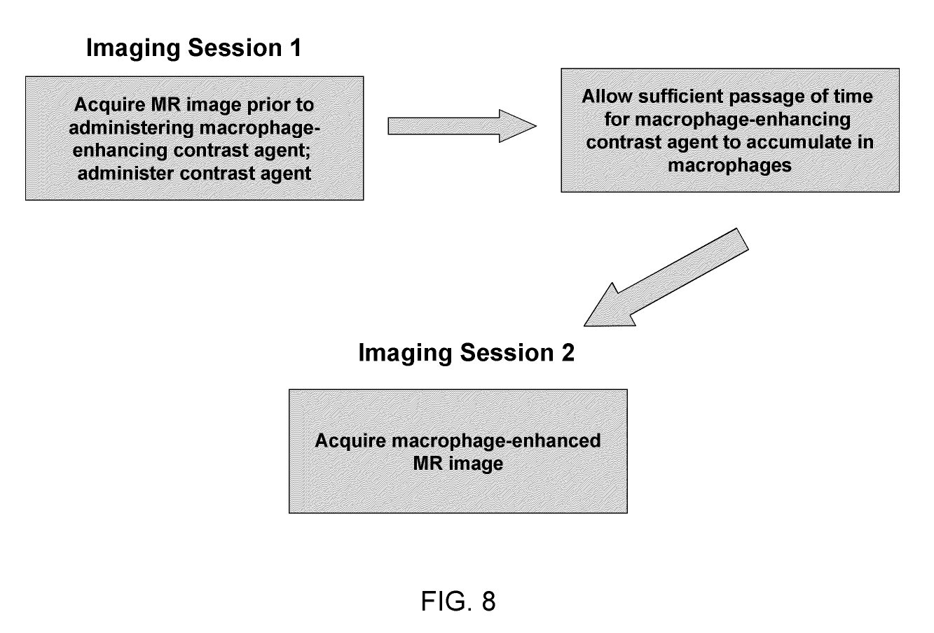

In particular embodiments, potential macrophage populations are imaged prior to administration of the macrophage-enhancing agent, followed by subsequent imaging when there is sufficient accumulation of the agent in the macrophage target. This paradigm is reflected in FIG. 8. When a contrast agent is first administered as a bolus injection, it passes through the vascular tree and resides primarily in the blood for a duration of time. Given a relatively constant blood flow rate, perfusion (a measure of the quality of vascular supply to a tissue, which is also a reflection of tissue activity) can be measured with MRI by coordinating the time of imaging of the desired tissue to the time when the bolus passes through it. MR images may be acquired before and after the contrast agent passes through the tissue of interest, and a comparison of the images produced before and after administration of the contrast is used to determine physiological characteristics. Various types of perfusion imaging (e.g., dynamic contrast-enhanced MRI) can provide information related to blood flow, vessel permeability and tissue volume.

After a short time of diffusion, the contrast agent achieves a relatively steady state concentration in the blood. This steady state is also useful for certain imaging modes. Certain contrast agents persist in the blood for a sufficient length of time such they function as blood pool agents. During this period, vascular-enhanced MRI modes may be performed, including, for example, black & bright blood imaging and magnetic resonance angiography (“MRA”).

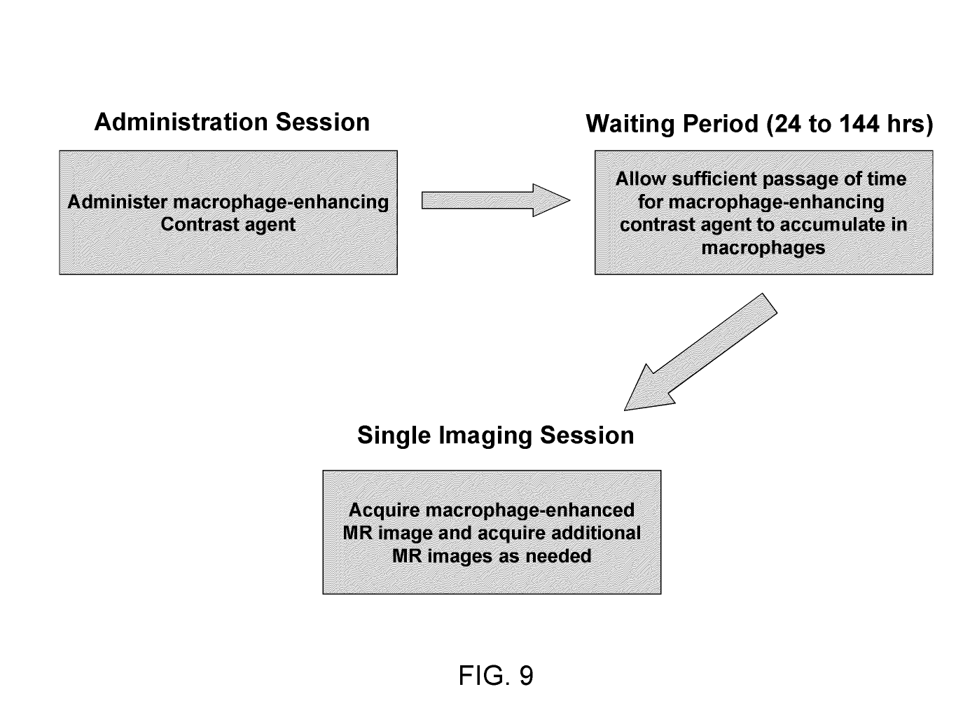

Macrophage-enhancing contrast agents slowly accumulate in macrophages over the course of several days. Particular embodiments of the present invention delay the imaging that would normally be performed at the time of administration of the contrast agent and instead waiting until macrophage enhancement has occurred. This paradigm is reflected in FIG. 9. With the long blood half-life associated with the contrast agents used in embodiments of the invention, a first magnetic resonance image can be obtained one or more days after administration of the contrast agent. The first magnetic resonance image may be immediately followed by acquisition of additional MR images, optionally in the presence of additional doses of a contrast agent. The additional MR images can provide information pertaining to physiological phenomena other than those observable with the MEMRI. A combination of imaging protocols in this manner can provide more powerful diagnostic capability than a single scan, since information can be made available in a single session relating to different physiological phenomena. Indeed, a pre-contrast agent MRI scan may not be required. Also importantly, all of the desired imaging can be completed in a single imaging session.

Macrophage enhancement, as described in this application, is based upon the ability of the biomarker to identify anatomic regions where normal macrophage populations are numerous, such as liver, spleen, lymph node and bone marrow as well as abnormal anatomic regions where accumulations of macrophages represents a pathophysiologic process. In the best studied class of useful biomarkers, the ultrasmall superparamagnetic nanoparticles, such as ferumoxtran-10, ferumoxytol, and ferucarbotran, their size and coating create a long-lived vascular distribution following administration. This is due to the very slow transit from the vascular space in regions that is characteristic of most of the body tissues. But there are normal tissues, such as liver, spleen, lymph node and bone marrow that have high vascular permeability. Macrophage populations in these tissues have access to the MEMRI biomarker and trap the effective agent for subsequent imaging. Some of the pathophysiological processes that are effectively imaged also have increased vascular permeability and this is facilitated by cytokines released from the local macrophages that have accumulated in the diseased tissues. The long vascular phase and limited vascular permeability sustains a vascular reservoir of biomarker for a sufficient time to allow the improved targeting of the effective MR biomarker to the macrophages in regions of high permeability for MEMRI.

In particular embodiments of the invention, a macrophage imaging contrast agent is administered to a subject by an operator or practitioner during an administration session. In certain embodiments of the invention, the macrophage imaging agent is a USPIO complex that is injected into the subject. Alternatively, the USPIO contrast agent may be administered orally. No imaging occurs during this session, so the session does not need to occur at an imaging center. In particular embodiments of the invention, the administration session may occur at a laboratory, doctor's office, outpatient clinic, hospital room or emergency room. Alternatively, the administration session may occur at a subject's residence or at the scene of a medical emergency or trauma.

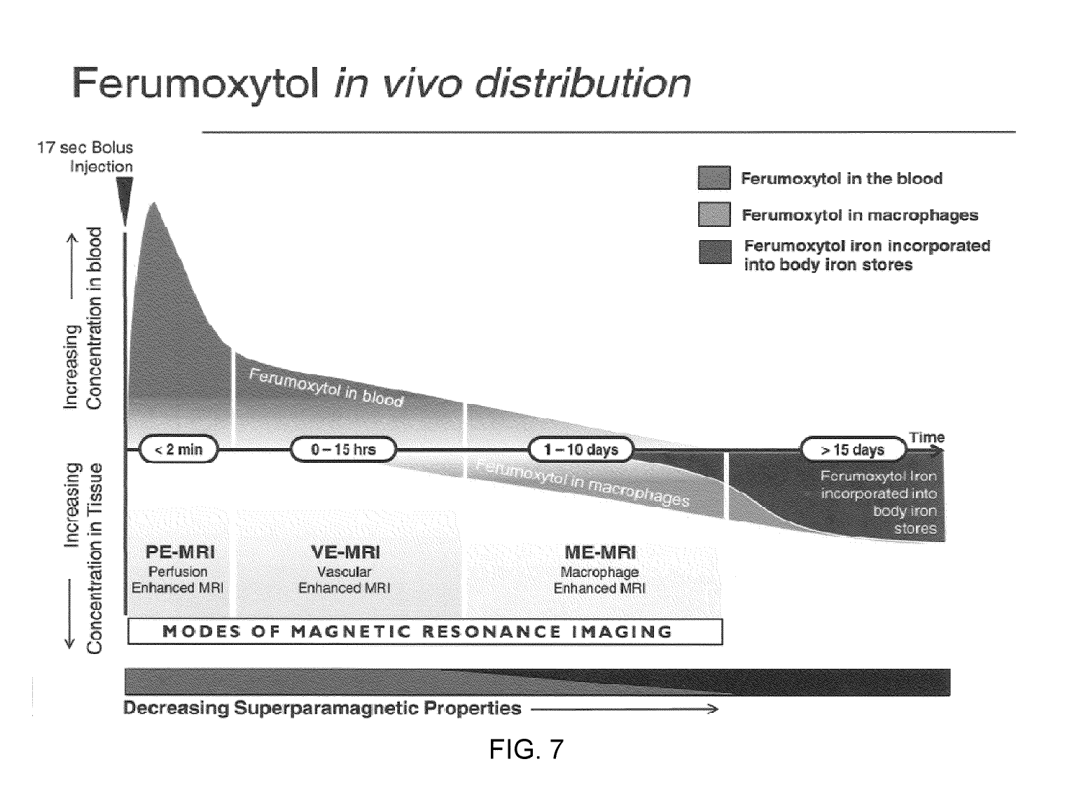

In particular embodiments of the present invention, following the administration session, there is a waiting period of 24 to 144 hours. In certain embodiments of the invention, the waiting period is between 48 and 96 hours. The waiting period provides time for the macrophage-enhancing contrast agent to clear from biological spaces (e.g., the vascular system into the interstitial spaces of the tissues), and accumulate in macrophages. At this time, the contrast agent will be near a maximal concentration in macrophages and minimal in blood, which provides an optimal condition for imaging during the MEMRI. See FIG. 7. A MEMRI is then performed at an imaging session. An operator or practitioner may then use the information obtained from the MEMRI image to provide a prognosis or predictive information, thereby defining a region of further imaging interest (e.g., a tumor, narrowed vessel, etc.).

Next, additional diagnostic information is provided in the same imaging session by performing a second MRI. See FIG. 9. The second MRI may be directed towards physiological phenomena or parameters other than macrophages. In certain embodiments of the invention, the second MRI is performed without administering any additional contrast agent. In these embodiments, the previously administered USPIO contrast agent is used as a single contrast agent. In an alternative embodiment, a second dose of a contrast agent may be administered. The second dose of contrast agent may be of the same USPIO contrast agent given at the administration session. Alternatively, the second dose of contrast agent may utilize a different contrast agent. The particular type of second MRI scan performed may include, but is not limited to: PEMRI, VEMRI, dynamic MRI, diffusion, MRA, multinuclear, and susceptibility weighted imaging. In alternative embodiments, the composite MRI may be utilized in image guided surgery or biopsy.

The second scans provide additional data on tissue pathology and physiology, but do so by recording physiological image data other than those pertaining to macrophages. In other words, the second MRI is not a MEMRI. For example, the second MRI may target the liver, spleen, bone, joints, lung, gastrointestinal tract, cardiovascular system, reproductive organs (e.g. ovaries, cervix or prostate), pancreas, kidneys, brain or any tissue afflicted with a chronic inflammatory disease. The second scans may also utilize perfusion-enhanced MRI techniques (“PEMRI”) during the second scan. PEMRI measure the rate at which blood is delivered to tissue and are sensitive to microscopic levels of blood flow. These measurements reflect, among other properties, the vascularization of the tissue. Tissue perfusion may be imaged via a T1-weighted gradient echo or an echo-planar imaging sequence after the application of an appropriate contrast agent.

T2* susceptibility effects may also be useful parameters in perfusion imaging. When T2*-susceptibility effects are imaged, the contrast agent causes a loss of MR signal, most marked on T2* (gradient echo)-weighted and T2 (spin echo)-weighted sequences, as a result of the magnetic field-distorting effects of a paramagnetic substance. T2* perfusion uses dynamic sequences based on multi or single shot techniques. The T2* (T2) MRI signal drop is caused by spin dephasing during the rapid passage of contrast agent through the capillary bed. The signal decrease is used to compute the relative perfusion to that region.

Both bolus tracking and arterial spin labeling MRI approaches may be utilized in the perfusion-enhanced MRIs of the present invention. Bolus tracking approaches utilize dynamic susceptibility contrast agents to obtain information about blood flow and volume. These techniques, also referred to as dynamic susceptibility contrast (“DSC”) MRI, involve an injection of a bolus of contrast agent and the rapid measurement of MR signal loss due to spin dephasing (i.e., decrease in T2 and T2*) during the fast passage of the bolus through the tissue. Coordinating the time of imaging of the desired anatomical feature to the time when the bolus passes through that feature, known as first-pass imaging, can provide useful information. A fast imaging technique may be utilized to obtain sequential images of the contrast agent as it moves through the tissue. Echo planar and potentially echo volume techniques together with appropriate computing power offer real-time images of dynamic variations in water characteristics reflecting perfusion and flow.

Arterial spin-labeling (“ASL”) methods for measuring perfusion are based on the fact that the magnetization and relaxation characteristics of tissue water can be affected by the flow of blood into the tissue. If the magnetic state of the blood water spins is different from that of the tissue water spins, an MRI sensitive to blood flow may be obtained. Subtraction of the spin-labeled image from a control image generates an image where the strength of signal is related to perfusion. In embodiments of the present invention, the control image may be the image generated from the MEMRI. Alternative embodiments of the invention may utilize continuous ASL or pulsed ASL techniques.

The second MRI scan may optionally be followed by one or more additional MRI scans. For example, the third MRI scan may target the liver, spleen, bone, joints, lung, gastrointestinal tract, cardiovascular system, reproductive organs (e.g. ovaries, cervix or prostate), pancreas, kidneys, brain, or any tissue afflicted with a chronic inflammatory disease. In certain embodiments of the invention, these additional MRI methods may be used to evaluate the vascular system; for example, the neuronal-, peripheral- or cardiovascular systems. These MR vascular-enhancing techniques (“VEMRI”) are able to evaluate the morphology and hemodynamics of the vascular system through imaging options that emphasize vascular visualization; for example, magnetic resonance angiography (“MRA”) to visualize moving blood. MR vascular imaging techniques utilize both spin echo and gradient imaging sequences. Spin echo imaging combines radiofrequency pulse combinations of differing degrees depending upon factors selected by a MRI operator. The pulse sequences may be supplemented with additional techniques such as gradient moment rephrasing, pre-saturation and double-inversion blood suppression. These techniques either enhance or void the signals from nuclei flowing in blood, thereby producing contrast between the vessels and the surrounding tissue.

Vulnerable plaques in an artery can be detected using an appropriate MR sequence known to those of skill in the art. For example, a fat-suppressed 2-dimensional T2*-weighted spectral-spatial excitation pulse may be used. Additional vascular anatomy structure contrast can be provided by black blood imaging (utilizing spin echo acquisitions to produce images where the blood is dark), bright blood imaging (utilizing gradient echo imaging and/or gradient moment rephrasing and/or contrast enhancement to make vessels appear bright), and time-of-flight MRA (produces vascular contrast by manipulating longitudinal magnetization of stationary spins).

If a second dose of contrast agent is administered during the imaging session, the second dose of contrast agent may be identical to or different from the contrast agent administered during the first visit, depending on the specific information that an operator or practitioner wishes to obtain. It may be a positive or negative contrast agent. Contrast agents for use in the second scan may include, for example, gadolinium chelates, ferumoxides, ferucarbotran, ferumoxtran, ferumoxytol, blood pool agents, ferric ammonium citrate, manganese chloride, ferumoxsil, and perfluoro-octylbromide. The second dose of contrast agent may be administered as a single injection or a series of injections. It may be rapidly injected in a bolus-type manner or it may be injected over the course of one or more acquisitions of images. In other embodiments of the invention, the second dose of contrast agent may be administered orally.

In one particular embodiment, a second dose of contrast agent is administered immediately following a MEMRI, and a PEMRI is performed within minutes of the second dose of contrast agent, thereby providing additional information at the same visit. In a further embodiment of the invention, a VEMRI may be performed if clinically indicated by the results of the MEMRI and PEMRI. The VEMRI may be performed after passage of a sufficient amount of time for the second contrast agent to reach steady state levels within the vasculature system. The intent of such a combination could be to image the thickness and shape of a fibrous cap in a plaque at risk of rupture causing a cardiovascular event such as stroke or myocardial infarction.

The composite MEMRI protocols may be utilized in the diagnosis of hyperacute and chronic disease or to study haemodynamic changes associated with any physiological state. In addition to the aforementioned applications to cancer and atherosclerosis, composite MEMRI protocols may assist in diagnosis or provide information on cerebral blood volume, stroke, ischemia and myocardial infarction.

Importantly, all of the MRI scans are capable of being performed at a single imaging session. Using the imaging protocols of particular embodiments of the invention achieves the unexpected result of eliminating additional imaging sessions while maintaining the functional and diagnostic capability of multiple imaging sessions. This finding saves time and resources, thereby allowing additional subjects to be imaged. Furthermore, the image protocols described herein achieve a distinct treatment advantage through the synergistic effects of the specific order in which multiple MRIs may be performed. It allows a practitioner to identify regions of interest prior to performing additional MRIs, thereby allowing the practitioner to focus the subsequent MRIs only on the region of interest. By tailoring the subsequent MRI techniques to the region of interest, a clearer image and improved prognostic information may be obtained. Particular value is achieved when the imaging outcome can be used in therapeutic decisions, including continuation of the therapeutic plan, modifying the plan, or even discontinuing portions of the treatment.

By taking advantage of this effect in embodiments of the present invention, the practitioner can (a) provide a more accurate assessment of a disease state, (b) determine whether additional MRI imaging is necessary (c) identify regions of interest for focused inquiry, and (d) customize subsequent MRI to the condition under evaluation, all while minimizing office visits and maximizing resources.

### EXAMPLE 1

### MEMRI Evaluation of Patient with Suspected Cancer in Single Breast

This situation involves a patient presenting with known or suspected cancer of one breast but a normal mammogram of the opposite breast. Recently, it has been suggested that such patients should undergo contrast-enhanced breast MRI to rule out other cancer foci (see Lehman, et al (2007) N Eng. J Med 356, 1295-303). In such an evaluation, the contrast agent is usually a gadolinium chelate and abnormal breast tissue is expected to show a focal accumulation of gadolinium in the expanded extracellular space associated with the cancer that was not clinically or mammographically evident. Though sensitive, this procedure is fraught with false positives—the abnormal regions must be biopsied and four of five such regions will not be cancerous—and does not provide information on the possible metastasis to local lymph nodes. In the present invention, the patient at risk is administered the macrophage biomarker and the breast and axilla are imaged with MRI at a time when macrophage labeling is evident—usually 12-168 hours, but preferably 24-72 hours later. Normal regional lymph nodes will accumulate the macrophage agent whereas nodal tissue replaced by metastasis will not. Most aggressive breast cancers or those with a poor prognosis show a region of accumulated excess macrophages. The presence of these macrophages is detected by the MRI examination. This detection is more specific than excessive gadolinium accumulation.

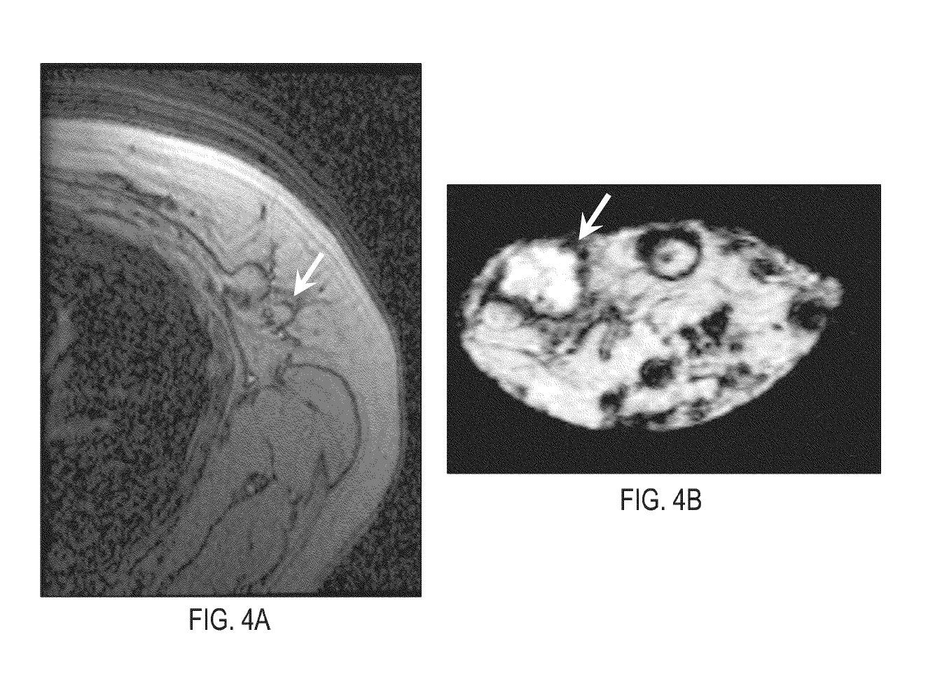

FIGS. 4A and 4B show a patient with breast cancer with macrophages around the primary tumor and displaced from the metastatic tumor in an adjacent lymph node tumor. FIG. 4A is an in vivo MRI of the patient's breast with a contrast agent of the invention; FIG. 4B is an in vitro MRI of the removed specimen containing the tumor and a metastatic lymph node, also with a contrast agent of the invention. The arrow in FIG. 4A shows the very clear presence of a dark accumulation of macrophages indicating a tumor. FIG. 4B shows the lymph node tumor indicated by a dark outline of macrophages where the center area of the tumor is light, because the cancer cells have displaced the macrophages from this central region of the tumor.

This patient was imaged following the administration of Combidex. The in vivo image in FIG. 4A identifies a primary breast tumor (arrow) and a metastatic lymph node tumor. The tissue was removed and a high resolution T2 weighted in vitro MRI performed. (FIG. 4B). With this MR pulse sequence, the macrophage enhancing agent is identified by the dark rim surrounding the primary breast cancer. Within the lymph node, normal macrophages similarly identified the lymph node tumor, but in this case there is a central zone where the macrophages have been displaced by metastatic cancer. Histopathology confirms the primary and metastatic tumors. This example shows the utility of identifying macrophages in regions where they represent pathology and the absences of macrophages from normal structures where they should be abundant.

If desired, other regions of the body can be imaged at the same time without an additional contrast administration to evaluate the presence of cancer in, for example, brain, lung, liver, or bone.

It is evident that gadolinium enhancement and macrophage enhancement can also be combined. Where desirable, the MEMRI exam is done as above, followed immediately or later by the gadolinium-enhanced MRI.

### EXAMPLE 2

### Patient with Breast Cancer and Bone Pain

When metastatic breast cancer is suspected, it is important to rule out the most common sites of metastasis, as well as recurrence or new cancer in the breast. If whole-body MRI is performed during macrophage enhancement, in other words, if a whole-body MEMRI is performed, identification of soft tissues where there is excessive macrophage density will identify where metastasis may be present. In addition, bone MRI is the best examination for bone metastasis, and the identification of macrophage dense areas by MEMRI would increase diagnostic accuracy in bone. Finally, displacement of normal macrophages in liver, spleen, or lymph nodes would suggest the presence of metastasis in these sites. One additional use of the macrophage imaging technique MEMRI is to identify regions where a tissue biopsy may be obtained for pathological information.

### EXAMPLE 3

### Patient with Bladder Cancer and/or Bone Pain

This example is similar to the above examples with breast cancer, with the additional advantage that regional nodes can be reexamined along with liver and lung, or other sites of potential metastasis.

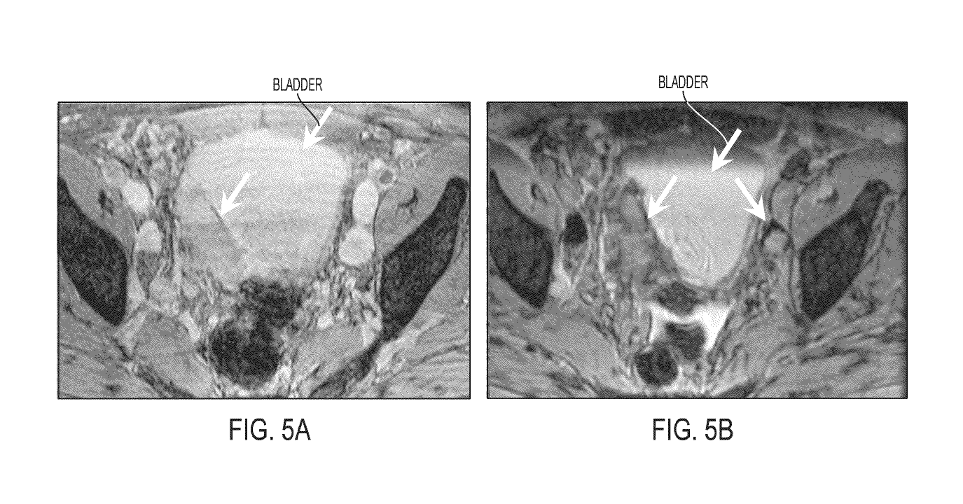

FIGS. 5A and 5B show a patient with bladder cancer with macrophages around the primary tumors. The bladder is indicated generally with an arrow in FIGS. 5A and 5B as the large central light area in the center of the pelvis region. FIG. 5A is the MRI without contrast agent and FIG. 5B is the image with contrast agent of the invention. In FIG. 5A, the tumor's presence is only hinted at by the “crease” in the bladder (shown with an arrow) that seems to be an indication of pressure on or displacement of the bladder along this juncture. FIG. 5B, with contrast agent, clearly shows the line of demarcation for the tumor along that “crease”, with the massive tumor showing as a dark mass directly to the left of this line, continuing down to a point and back again. A second smaller tumor is indicated with an arrow to the right of the bladder, appearing as a bulls eye type node. This second tumor is outlined with a dark ring of macrophages. The center of the tumor shows up lighter where the cancer cells have displaced the macrophages. As an indication of the power of this contrast agent in cancer diagnosis and staging, MRI prior to MEMRI (FIG. 5A) merely hints at a large lesion adjacent to the bladder. Following MEMRI (FIG. 5B) the lesion is seen to be large with a substantial content of macrophages and invasion of the bladder wall. The macrophage content suggests a high degree of angiogenicity and likely aggressive local tumor growth.

Once diagnosis of the primary tumor is made, whole-body MRI is appropriate. If a whole-body MEMRI is performed, identification of soft tissues where there is excessive macrophage density will identify where metastasis may be present. In addition, bone MRI is the best examination for bone metastasis, and the identification of macrophage dense areas by MEMRI would increase diagnostic accuracy in bone. Finally, displacement of normal macrophages in liver, spleen, or lymph nodes would suggest the presence of metastasis in these sites. One additional use of the macrophage imaging technique MEMRI is to identify regions where a tissue biopsy may be obtained for pathological information.

### EXAMPLE 4

### Patient with Aggressive Prostate Cancer and Possible Bone Pain

This example is similar to the above examples with breast cancer, with the additional advantage that regional nodes can be reexamined along with liver and lung, or other sites of potential metastasis.

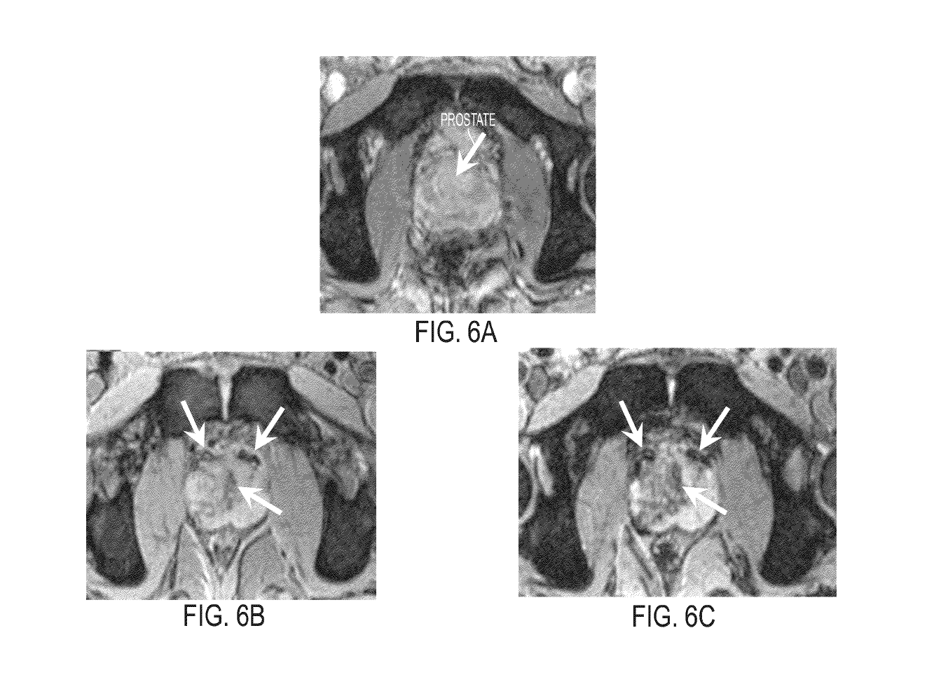

FIGS. 6A, 6B and 6C show MRI depictions of a patient with prostate cancer. The prostate is indicated generally as the large central circular space in the center of the pelvis region. FIG. 6A is the MRI without contrast agent, and FIGS. 6B and 6C are the MRIs with a contrast agent of the invention. The presence of a tumor is not indicated at all in FIG. 6A, the MRI without contrast agent. In stark comparison, FIGS. 6B and 6C indicate the presence of a very large tumor, and possibly multiple large tumors, within the prostate, as indicated by the three arrows pointing out regions of the tumor (or tumors) that are particularly enhanced with macrophage, in the presence of a contrast agent of the invention. In FIG. 6C, one can more clearly see the seemingly massive size of the tumor, as well as its amorphous nature (indicated by an arrow to the central left portion of the prostate), where macrophage have infiltrated the tumor and cause the tumor to appear mottled dark and light grey in this image.

The MRI prior to MEMRI (FIG. 6A) shows an enlarged prostate gland with little cellular discrimination. Following MEMRI (FIG. 6B and 6C), the prostate cancer is seen to include multiple zones with surrounding macrophages. This finding is believed to reflect poor prognosis.

Once diagnosis of the primary tumor is made, whole-body MRI is appropriate. If a whole-body MEMRI is performed, identification of soft tissues where there is excessive macrophage density will identify where metastasis may be present. In addition, bone MRI is the best examination for bone metastasis, and the identification of macrophage dense areas by MEMRI would increase diagnostic accuracy in bone. Finally, displacement of normal macrophages in liver, spleen, or lymph nodes would suggest the presence of metastasis in these sites. Again, an additional use of the macrophage imaging technique MEMRI is to identify regions where a tissue biopsy may be obtained for pathological information.

### EXAMPLE 5

### Patient with Metastatic Disease Expressing Excess Macrophage Density Undergoing Treatment

Prior to the initiation of chemotherapy with expected dose-related side effects, the sites of metastases are determined with delayed macrophage-enhanced MRI (MEMRI). As chemotherapy progresses, the reduction in macrophage density indicates efficacy whereas the unabated presence of the same or increased macrophage density indicates incomplete therapeutic response.

### EXAMPLE 6

### General Whole-Body MEMRI Protocol

Either T1-weighted and fast-spin echo T2-weighted images, complimented with gradient-recalled-echo (GRE) T2*-weighted sequences, or T2 and T2*-weighted sequences, are examples of imaging methods that are used with a suitable USPIO. Depending on the particular USPIO chosen, however, T1-weighted sequences alone may be sufficient. To capture a primary tumor and possible associated metastatic tumors, high resolution images are essential. Therefore, preferably, acquired images have a resolution of at least about 1-3 mm isotropic ideally, with at least 2-5 mm through plane, nominally. Siemens Medical Solutions and TIM (total imaging matrix) technology is one example system that may be utilized to acquire such high resolution images with a suitable USPIO such as ferumoxtran-10. Other useful imaging systems include the PoleStar N-10 system (Odin Medical Technologies, Yokneam Elit, Israel), the Magnetom Vision system (Siemens), the Sonata System (Siemens) using a rolling table platform (Body SURF, MR Innovation, Essen, Germany) and the Horizon system (GE Medical Systems).

As macrophage-enhancing contrast agents such as ferumoxytol slowly escape from the blood vessels after administration over the course of 12 to 168 hours or more, they leak into the interstitial space. They encounter monocytes that have been recruited through cytokine signals to the tumor or atherosclerotic lesion and have been differentiated into macrophages. It is there that the macrophages will internalize the contrast agent, which effectively acts as a biomarker, enabling imaging of these tumor-associated or plaque-associated macrophages. The sessile macrophages will also accumulate the MEMRI agent.

On T2- or T2*-weighted images, for normal tissues of the lymph node, liver and spleen appear as dark voxels because of the biomarker uptake by the macrophages, whereas voxels containing malignant tissues appear brighter due to lack of uptake of the particles by the tumor cells. Such images are referred to as “displacement images”, and the process is sometimes also referred to by us as negative MEMRI evaluation because the normal cells are displaced by the tumor and only the normal sessile macrophages are directly imaged by the USPIO biomarkers. By contrast, the T2- or T2*-weighted images for malignant tumors in other tissues will be identifiable by a darker voxels due to tumor-associated macrophages which have accumulated the USPIO. For atherosclerotic plaques, the USPIO accumulates within the plaque-associated macrophages, thereby shortening the local T2 and T2* relaxation rates and causing signal loss on the MR images.

In one embodiment, non-contrast enhanced T1-weighted and T2-weighted sequences may be taken using, for example, a section width of about 7 mm. Repetition times and echo times are, for example, 124 ms and 1.8 ms, respectively, for the T1-weighted sequences, and 1200 ms and 60 ms, respectively, for the T2-weighted sequences. Subsequently, the USPIO is administered to the patient, and after approximately 12-168 h, 5-10 successive, contrast-enhanced 3-dimensional data sets are acquired as the patient is moved through the imaging cavity. With certain advances, the whole-body MRI can be acquired continuously.

It is also possible to perform MEMRI evaluations of isolated, or partial regions of the body, such as of the torso, the legs, or excluding the head and neck, etc., as needed or indicated, and as instructed by the physician.

In another embodiment, the USPIO is administered to the patient at a dose of at least 2 mg/kg or up to the maximal safe dose, and a MRI is scheduled for 48-144 hours later, preferably 48 to 96 hours later. During the subsequent MEMRI visit, macrophages in a relevant body part can be detected with T2 or T2* whole-body MRI performed by choosing the voxel dimensions as appropriate. Macrophage accumulation in a relevant body part can be detected in any artery using an appropriate T2/T2* MR sequence. Similarly, tumor associated macrophages that have accumulated the USPIO may also be identified.

### 7. Staging Cancer and Providing a Prognosis Using MEMRI

A patient identified as having a malignant tumor (such as by clinical exam, other imaging or biopsy) is administered a macrophage biomarker and the suspected primary is tumor imaged with MEMRI, as described in Example 5, at a time when macrophage labeling is evident—usually 12-168 hours, but preferably 24-72 hours later. TAMs will accumulate the macrophage-seeking biomarker agent in tumor sites and so these sites will thus be visible by MEMRI. Depending on the morphology of the primary tumor and the presence, number and morphology of metastatic tumors, a physician can stage the cancer. The presence of TAMs indicates sites of tumor growth, and the density of the TAMs at the sites of tumor is an indication of tumor prognosis. Aggressive cancers and/or those with a poor prognosis show a region of accumulated excess macrophages. The presence of these macrophages is detected by the MRI examination and provides an indication of tumor stage and prognosis.

Multiple regions of the body can be imaged in this way, without administration of an additional contrast agent at the same time, to evaluate the presence of cancer in tissues such as, for example, breast, brain, lung, liver, muscle or bone.

Gadolinium enhancement and macrophage enhancement can also be combined, when desirable, in which case the MEMRI exam is done as above, followed immediately or later by the gadolinium-enhanced MRI. These same techniques can be used to identify and assess the metastatic potential of cancer foci.

### 8. Determination of Individualized Anticancer Therapy and Assessment of Anticancer Therapy Using MEMRI Evaluation

A patient identified as having a malignant tumor (such as by biopsy) is administered a macrophage biomarker and the patient is imaged with MEMRI, as described in Example 5, at a time when macrophage labeling is evident—usually 12-168 hours, but preferably 24-72 hours later. TAMs will accumulate the macrophage-seeking biomarker agent at tumor sites and these sites will thus be visible by MEMRI. Depending on the morphology of the primary tumor and the presence, number and morphology of metastatic tumors, a physician can determine the aggressiveness of the cancer and whether it has a good or a poor prognosis, based on the density of TAMs visible with the initial MEMRI, with tumors having a poor prognosis exhibiting a region of accumulated excess macrophages. The presence of these macrophages is detected by the MRI examination and provides an indication of tumor stage and prognosis. This, in turn, is used to determine an individualized anticancer therapy for that patient, which may comprise chemotherapy, radiation therapy, surgery, and immunotherapy, in various combinations, sequences, or alone.

Then, at predetermined times during and after the anticancer therapy, follow-up MEMRI evaluations may be directed performed, as directed by the physician, and compared to the base-line MEMRI evaluation performed prior to the anticancer therapy. A decrease in the number of tumors or the size of the tumors, as evidenced by the observance of TAMs using MEMRI, or evidence that the macrophage density and displacement associated with a primary cancer or metastatic cancer shows regression or is progression free in the post-treatment image compared to the pre-treatment image is evidence of the efficacy of the anticancer therapy. In contrast, evidence that the number of tumors or the size of the tumors is increasing, based on the observance of TAMs using MEMRI, or evidence that the macrophage density and displacement associated with a primary or metastatic tumor is still progressing, post-treatment, instructs the physician to modify or suspend the current anticancer therapy in favor of an alternate/additional treatment regimen.

As described above, multiple regions of the body can be imaged in this way, without administration of an additional contrast agent at the same time, to evaluate the presence of cancer in tissues such as, for example, breast, brain, lung, liver, muscle or bone. In addition, as described above, gadolinium enhancement and macrophage enhancement can also be combined, when desirable, in which case the MEMRI exam is done as above, followed immediately or later by the gadolinium-enhanced MRI to establish baseline scans, after which the anticancer therapy is administered, and follow-up MEMRI and gadolinium-enhanced MRI is again performed and compared to the pre-treatment images.

These same techniques may also be adapted to provide in a single imaging session real-time feedback on the efficacy of an anti-cancer treatment such as chemotherapy. For example, 24-72 hours after a microphage-imaging-agent administration session, a subject is imaged with MEMRI to establish a pre-treatment image. The pre-treatment image provides a baseline assessment of TAM density surrounding a primary or metastatic tumor of interest. Immediately following the MEMRI, the subject undergoes a cancer treatment session comprising chemotherapy regimens known to those of skill in the art, optionally including. Examples of particular chemotherapy regimens include CMF (cyclophosphamide, methotrexate, fluorouracil), m-BACOD (methotrexate, bleomycin, adriamycin (doxorubicin), cyclophosphamide, Oncovin (vincristine), dexamethasone)), MVAC (methotrexate, vinblastine, adriamycin, cisplatin), antifolates, folate analogues, other dihydrofolate reductase inhibitors, and variations of any of the foregoing known to those of skill in the art. The cancer treatment session can also include spatially targeted treatments directed specifically towards the tumor of interest; for example, radiation therapy, targeted drug delivery or liposomal drug delivery. The cancer treatment can further include extirpation, in situ ablation, radiotherapy, immunotherapy, gene therapy and alternative therapy

During or immediately subsequent to the cancer treatment session, the subject undergoes a second MEMRI directed towards the primary or metastatic tumor or region of the body afflicted by cancer. The second MEMRI establishes a post-treatment image and provides a measure of TAM density immediately following the cancer treatment. Comparison of the pre-treatment image to the post-treatment image provides a near real-time provisional assessment on the effectiveness of the treatment. For example, the second MEMRI may allow for assessment of the accuracy of the spatially targeted treatments. In this manner, the subject undergoes a combined single imaging/cancer treatment session capable of providing immediate feedback regarding the effectiveness or appropriateness of a cancer treatment. In addition, gadolinium enhancement and macrophage enhancement can also be combined when desirable, as described above.

These same techniques may be used to determine the frequency of follow-up MEMRI evaluations in a subject, to assess ongoing treatment, determine whether the patient is in remission, determined whether a secondary cancer has emerged in a patient, and/or look for metastasis, among other things.

### 9. Use of MEMRI to Determine a Site for Biopsy

A patient identified as being at risk for a malignant tumor because of physical indicators and evidence, such as observation of a strange mole, lump, pain, or other indicators, is administered a macrophage biomarker and the patient is imaged with MEMRI, as described in Example 5, at a time when macrophage labeling is evident—usually 12-168 hours, but preferably 24-72 hours later. TAMs will accumulate the macrophage-seeking biomarker agent at tumor sites and thus be visible by MEMRI. Depending on the morphology of the observed tumors and the presence, number and morphology of possible metastatic tumors, a physician determines a site for biopsy. Potential biopsy sites will be chosen, for example, if there is evidence of one tumor being the primary tumor. Such evidence may include an accumulation of TAMs at one suspected tumor site over another, as observed by MEMRI evaluation. Other evidence may be the morphology, size and position of a suspected tumor, as observed by MEMRI. Since the presence of TAMs indicates sites of tumor growth, the density of the TAMs at a given site will enable a physician to determine a site for biopsy. Aggressive cancers and/or those with a poor prognosis show a region of accumulated excess macrophages. In addition to providing a physician to determine a site for biopsy, the presence of tumor-associated macrophages is detected by the MRI examination through the use of macrophase-seeking biomarkers, and so MEMRI evaluation also provides an indication of tumor stage and prognosis, once the biopsy confirms that the tumor is malignant. The biopsy so obtained from a region with active TAMs may be analyzed for genetic or compositional information that may inform therapy.

Multiple regions of the body can be imaged in this way, without administration of an additional contrast agent at the same time, to evaluate the presence of cancer in tissues such as, for example, breast, brain, lung, liver, muscle or bone. Gadolinium enhancement and macrophage enhancement can also be combined, when desirable, in which case the MEMRI exam is done as above, followed immediately or later by the gadolinium-enhanced MRI. These same techniques can be used to identify and assess the metastatic potential of cancer foci.

### EXAMPLE 10

### Use of a Report Card to Follow Up Treatment

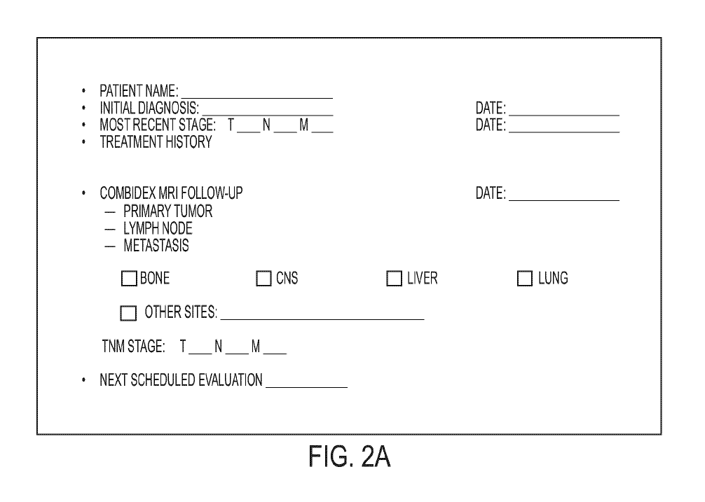

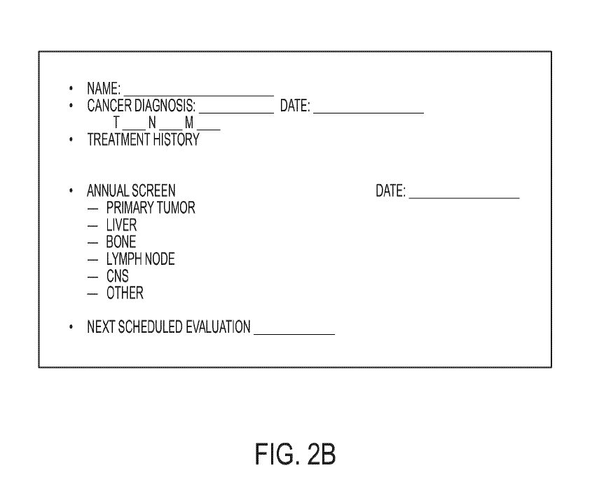

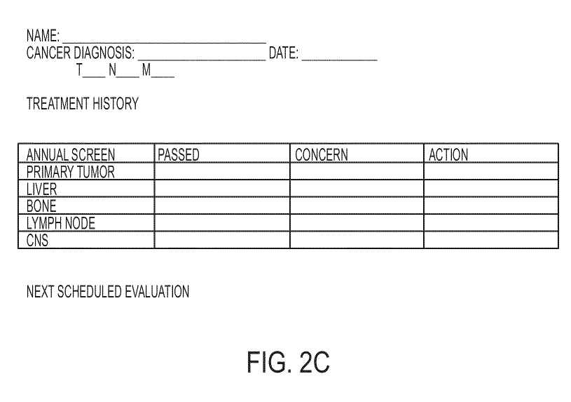

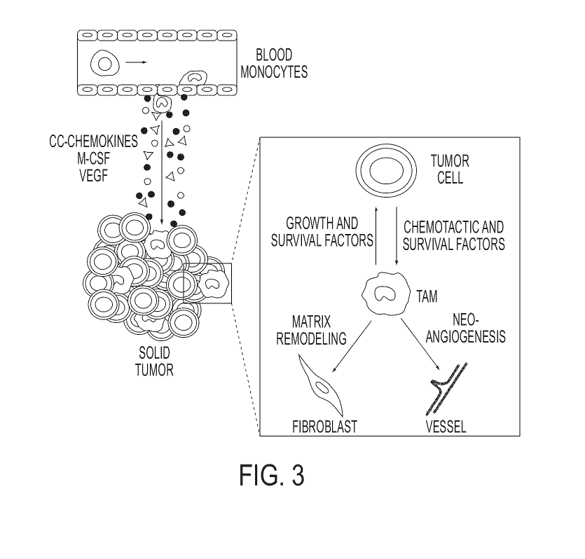

As an aid to a physician in assessing and following treatment for a given cancer patient, a report card, such as shown in FIGS. 2A, 2B and 2C made be used. The report card may include fillable spaces for patient information, the date original and new information is entered, spaces for information and date regarding an initial MEMRI, and for the next scheduled MEMRI evaluation and all follow-on MEMRI evaluations. A report card may also contain fillable spaces for information relating to the initial diagnosis, information relating to the initial stage or staging of the cancer, for information relating to the nature or cell type of the primary tumor, additional space for information relating to secondary tumors found or suspected, space for adding information relating to follow-up MEMRI evaluation information, and fillable space for information relating to standard TNM Stage procedures. Of course, any combination of categories are envisioned for such a physician report card, as well as additional categories for the report card, depending on the patient, the nature and type of cancer, and the needs of the physician. For example, the report card may be organized and/or designed to aid primarily the patient. In such an embodiment, the report card serves to provide information and a succinct summary or snapshot of the ongoing progress of the patient's disease and treatment plan, prognosis, in layperson's terms and designed to provide information that will be helpful and informative from the patient's point of view. Other embodiments may be organized and/or designed to aid primarily the physician and healthcare providers, providing a similar succinct summary or snapshot of the ongoing progress of the patient's disease and treatment plan, prognosis, but presented more technically and clinically, i.e. such a report card would be designed to include information helpful and informative from a physician's or other healthcare provider's point of view.

The embodiments of the invention described above are intended to be merely exemplary; numerous variations and modifications will be apparent to those skilled in the art. All such variations and modifications are intended to be within the scope of the present invention as defined in any appended claims.

### EXAMPLE 11

### Composite Biomarker Protocol

During a first visit, a subject is given an appropriate dose of ferumoxytol. Preferably, an MRI evaluation is then scheduled for 48-96 hours later.

During a second visit or imaging session, a MEMRI image of the relevant body parts is obtained to visualize enhanced macrophages using T2/T2* sequences. If the results of the MEMRI indicate the clinical desirability of obtaining further information, a PE-MRI may be performed. An additional low dose of ferumoxytol may be given at this second visit for use in dynamic susceptibility imaging (PEMRI) of the region(s) of interest. This step may be repeated as needed. Optionally, it is also possible to image the vascular lumen at the same visit with a blood-enhancing technique. Additionally, the composite protocol may include a non-contrast technique or contrast-enhanced techniques utilizing administration of gandolinium or USPIO agents. A VEMRI may also be performed at the second visit if clinically indicated. For example, a suitable dose of ferumoxytol is given and a steady-state MRI performed to visualize arterial and venous anatomy and to measure regional blood volume.

One benefit of the composite protocols of embodiments of the invention, wherein a MEMRI is performed before any other MRI techniques, is that the MEMRI image data permit wiser use of additional MRI techniques—in a single imaging session—by enabling intelligent selection of additional MRI procedures to be performed. After the MEMRI is completed, all MR imaging techniques known to those of skill in the art remain available for precise diagnosis or monitoring of disease. The MEMRI data enable selection of the best of these techniques available to a practitioner, a circumstance enabling intelligent and comprehensive diagnosis within a single, efficient imaging session.

### EXAMPLE 12

### Breast Cancer Staging

A woman with a high risk history and a palpable lump in the right breast with a suspicious mammogram is referred for preoperative MR staging. On a first visit, she is given 5 mg/kg ferumoxytol and scheduled for an MRI at a second visit 4 days later. On the second visit, a bilateral breast MRI with T2* pulse sequences, including the regions of the axillary, subclavian, and sterna lymph nodes, is performed to determine nodal grade, and macrophage density surrounding all identified breast cancers.

Options for additional MR scanning on this second visit include the following:

If a high grade tumor is detected or nodal metastasis is seen, the liver and bone marrow may be imaged with MR to determine if metastasis has occurred and to provide a baseline for subsequent follow up. Additional MR detail can be obtained as necessary using an additional dose of ferumoxytol or gadolinium-based contrast agents to assess tumor angiogenesis.

Other MR studies of established utility may also be performed at this time. These could include, for example, diffusion-weighted MR or MR spectroscopy. Again, it is to be expected that the imaging parameters used for the additional MR imaging and the interpretation and subsequent results of the imaging resulting therefrom would be guided by the results that have previously been obtained from the MEMRI.

One advantage of the composite MR imaging protocols of embodiments of the invention is that results of the MEMRI allow subsequent imaging activities to be focused on a particular region of interest, thereby producing superior imaging capabilities. Following the MEMRI, subsequent imaging techniques can be appropriately selected and guided by the results produced from the MEMRI.

These additional MR studies are not an inclusive list but are shown as examples of combined MR scanning that can be performed as needed in the single session that includes MEMRI assessment.

### EXAMPLE 13

### Breast Cancer Restaging

Periodically for active surveillance, or when metastasis is clinically suspected, the MR imaging options described above can again be utilized, this time with whole-body MRI or with regional MRI of metastatic regions at risk, such as liver, lung, brain, lymph nodes and bone.

### EXAMPLE 14

### Breast Cancer Therapy Assessments

There are multiple chemo and radiation treatments that could be selected for individual patients. If effective, these treatments would be expected to first influence the macrophage density at tumor sites or the associated angiogenesis. Either or both of these are assessable with the composite MRI protocols of the embodiments of the invention. Such assessments may be qualitative, semi-quantitative or quantitative. These can be repeated as needed, providing a diagnostic opportunity that is especially favored, due to the lack of ionizing radiation.

### EXAMPLE 15

### Experimental Therapies in Breast Cancer

Methods of composite MRI protocols of embodiments of the invention may be included in clinical trials of new therapies. These might include studies from Phase 0 to Phase IV and would be expected to add safety and efficacy to such trials. Both tumor vascularity and tumor-associated macrophage density are expected to be more sensitive measures of tumor response—whether favorable or insufficient—than changes in tumor dimensions.

### EXAMPLE 16

### Vulnerable Plaque Assessment

The appropriate dose of ferumoxytol is given intravenously and MEMRI performed 1-10 days later. Regional or whole-body MEMRI identifies the arterial regions with active macrophage populations. Atherosclerosis is marked by an increase in endothelial cell permeability, facilitating accumulation of ferumoxytol in plaque-associated macrophages (foam cells), which causes a strong T2* effect leading to a focal signal void. Ferumoxytol uptake is high in rupture-prone lesions. Subsequently, other MRI (white blood, time of flight or arterial spin labeled or contrast-enhanced MRA) can be performed to identify the blood pool at those locations, thus quantifying the local stenosis. In order to image the important fibrous membrane overlying this plaque, it is desirable to perform high spatial resolution steady state VEMRI. The combination of macrophage enhancement plus blood pool enhancement serves to characterize the risk of plaque rupture with subsequent adverse cardiovascular events in the region served by that artery. Regions with ruptured plaque may be identified by the associated filling defects.

In a particular embodiment, an MEMRI with ferumoxytol indicates an active macrophage population in a carotid artery of a subject; for example in the atherosclerotic-prone geometry of the carotid bulb at the intersection of the common carotid artery, external carotid artery and internal carotid artery. In the same imaging session, the affected area is imaged in the tangential and longitudinal planes, according to previously published techniques through the use of gradient echo breath-hold acquisition. See, e.g., Hundley, et al., “Noninvasive determination of infarct artery patency by cine magenetic resonance angiography”, CIRCULATION, 91: 1347-1353 (1995). These images allow for visual assessment of luminal diameter stenosis. Information regarding the severity of vascular disease is also derived from measurements of flow through the vessel. In the same imaging session, flow is measured by cine phase—contrast breath-hold acquisition acquired perpendicularly across vessel segments in the cross-sectional slice position determined from the gradient-echo acquisitions. Imaging parameters and coronary arterial flow calculations are as described previously (Hundley, et al., “Assessment of coronary arterial restenosis with phase-contrast magnetic resonance imaging measurement of coronary flow reserve”, CIRCULATION, 101:2375-2381 (2000); Hundley, et al., “Assessment of coronary arterial flow and flow reserved with magnetic resonance imaging”, CIRCULATION, 93: 1502-1508 (1996); Clarke, G. C., et al., “Measurement of absolute epicardial coronary arterial flow and flow resererve using breath-hold cine phase-contrast magnetic resonance imaging”, CIRCULATION, 91: 2627-2634 (1995)).

Advanced atherosclerotic plaques are marked by neovascularation. Neovascularization within plaques represents a pathway for the recruitment of macrophage infiltration, and plaque neovascularization is recognized as playing an important role in pathogenesis. The presence of neovascularization is also associated with plaque instability and a heightened risk of plaque rupture. Neovascularization and increased endothelial permeability are hallmarks of plaque inflammation, which permits gadolinium-enhanced visualization of the vessel wall to function as a marker of inflammation. When indicated by the MEMRI, dynamic contrast—enhanced MRI is used to measure the rate of uptake of gadolinium-based contrast as described (Padhani, A. R. “Dynamic contrast-enhanced MRI in clinical oncology: current status and future directions”, J. MAGN. RESON. IMAGING, 16: 407-422 (2002)), thereby providing a quantitative measure of plaque inflammation.

### EXAMPLE 17

### Inflammatory Bowel Disease

A 28 year old woman with no history of inflammatory bowel disease presents to a clinician with abdominal pain and diarrhea. An intravenous dose of at least 2 mg/kg or up to the maximal safe dose of a USPIO is given, and a MRI is scheduled for 48-144 hours later, preferably 48 to 96 hours later. During the subsequent MEMRI visit, a T2 or T2* abdominal-focused MRI indicates accumulation of macrophages within beneath the surface epithelium of the intestines in the ileal and colonic mucosa, particularly near blood vessels. Subsequently and at the same imaging session, cross-sectional imaging techniques known to those of skill in the art are implemented to allow visualization of the entire bowel, without overlapping bowel loops. Magnetic resonance enterography and/or MR enteroclysis protocols are performed with oral administration of T1 or T2 contrast agents. An axial balanced steady-state free precession MR protocol shows ilial thickening, an irregular mucosal border consistent with ulcerations, and an intrauterine pregnancy. Various imaging protocols may be administered as described in Fidler et al., “MR Imaging of the Small Bowel”, RADIOGRAPHICS, 29:1811-1825 (2009), hereby incorporated by reference. Where clinically indicated by the results of the MEMRI, a volumetric interpolated breath-hold examination (“VIBE”; Rofsky, N. M. et al., “Abdominal MR imaging with a volumetric interpolated breath-hold examination”, RADIOLOGY 212: 876-884 (1999)) technique is used to reduce acquisition time by employing a combination of asymmetric sampling along the section-select gradient and sinc interpolation (zero filling) in k-space. This technique allows extremely thin sections and nearly isotropic voxels on the order of 2 mm to be obtained, which minimizes partial volume effects and allows for multiplanar reconstructions in any desired plane. This approach can distinguish between various forms of inflammatory bowel disease, detect complications such as penetrating disease and small-bowel obstruction, and may also be useful for monitoring disease activity (e.g., bowel wall thickening, hyperenhancement, ulcerations, increased mesenteric vascularity, and perienteric inflammation), response to therapy, or even as an efficacy outcome for experimental therapies.

What is claimed is:

1. A method of obtaining a series of enhanced magnetic resonance images in a single imaging session with a subject, the method comprising:
administering a macrophage imaging agent to the subject during an administration session;
allowing a passage of time sufficient for accumulation of the agent in macrophages of the subject; and
in a single imaging session:
(i) acquiring a macrophage-enhanced magnetic resonance image to target the macrophages; and
(ii) acquiring a different magnetic resonance image targeting a physiological phenomenon other than macrophages.

2. A method according to claim 1, wherein acquiring the different magnetic resonance image follows the acquisition of the macrophage-enhanced magnetic resonance image.

3. A method according to claim 2, further comprising administering a contrast agent before acquiring the different magnetic resonance image.

4. A method according to claim 1, further comprising using the macrophage-enhanced magnetic resonance image to observe macrophage activity associated with a primary tumor or metastatic tumor.

5. A method according to claim 1, further comprising using the macrophage-enhanced magnetic resonance image to observe macrophage activity associated with an atherosclerotic plaque.

6. A method according to claim 1, wherein the macrophage imaging agent is an ultrasmall superparamagnetic iron oxide particle.

7. A method according to claim 6, wherein the macrophage imaging agent is ferumoxtran-10.

8. A method according to claim 6, wherein the macrophage imaging agent is ferumoxytol.

9. A method according to claim 6, wherein the ultrasmall superparamagnetic iron oxide particle comprises a carboxyalkylated reduced dextran iron oxide complex.

10. A method according to claim 1, wherein sufficient passage of time for accumulation of the agent in macrophages is 24-144 hours.

11. A method according to claim 1, wherein the different magnetic resonance image targets the vasculature or perfusion.

12. A method according to claim 3, wherein the physiological phenomenon is perfusion.

13. A method according to claim 3, wherein the different magnetic resonance image targets the vasculature.

14. A method according to claim 3, wherein the contrast agent is selected from a group consisting of ultrasmall superparamagnetic iron oxide particles, superparmagnetic iron oxide particles, gadolinium compounds, manganese compounds, and perfluorocarbons.

15. A method according to claim 1, further comprising a third magnetic resonance image targeting a physiological phenomenon other than macrophages or perfusion.

16. A method according to claim 15, wherein the physiological phenomenon other than macrophages or perfusion comprises the vasculature.

17. A method according to claim 1, wherein the macrophage-enhanced magnetic resonance image comprises a whole-body MRI scan.

18. A method of assessing a stage of cancer in a single imaging session with a subject, the method comprising:
administering a macrophage enhancing contrast agent to the subject during an administration session;
allowing passage of time sufficient for accumulation of the agent in macrophages of the subject;
acquiring a first magnetic resonance image of regions of the subject's body at risk of cancer;
using the image to assess macrophage density and displacement associated with any primary cancer or metastatic cancer in the subject, such density and displacement being indicative of neoplasia; and
acquiring a second magnetic resonance image of the regions of the subject's body indicating neoplasia to measure a physiological phenomenon other than macrophages.

19. A method according to claim 18, wherein the macrophage enhancing contrast agent is ferumoxytol.

20. A method according to claim 18, wherein the passage of time is four days.

21. A method according to claim 18, wherein the cancer is breast cancer.

22. A method according to claim 21, wherein the regions of the body comprise the breast and axillary, subclavian and sterna lymph nodes.

23. A method according to claim 18, wherein the second magnetic resonance image is acquired from liver and bone marrow.

24. A method according to claim 18, further comprising acquiring at least a third magnetic resonance image following administration of a second contrast agent to assess tumor angiogenesis.

25. A method according to claim 24, wherein the second contrast agent is the same composition as the macrophage enhancing contrast agent.

26. A method according to claim 18, further comprising acquiring a diffusion-weighted magnetic resonance image or performing magnetic resonance spectroscopy.

27. A method according to claim 18, further comprising restaging the cancer at a later time.

28. A method of characterizing vulnerable atherosclerotic plaques in a single imaging session with a subject, the method comprising:
administering a macrophage imaging agent to the subject during an administration session;
allowing passage of time sufficient for accumulation of the agent in macrophages of the subject; and
in a single imaging session:
(i) acquiring a first magnetic resonance image to observe active macrophage populations in a region of the subject's vasculature susceptible to atherosclerosis; and
(ii) if an active macrophage population is observed, acquiring a second magnetic resonance image of the region to visualize a phenomenon other than macrophages.

29. A method according to claim 28, wherein acquiring the second magnetic resonance image includes doing so by bright blood imaging or magnetic resonance angiography.

30. A method according to claim 28, further comprising acquiring a high spatial resolution, steady state vascular-enhanced magnetic resonance image.

31. A method according to claim 28, wherein the passage of time is 1-10 days.

32. A method according to claim 1, wherein the single imaging session provides information for identification, localization and characterization of inflammatory bowel disease.

33. A method according to claim 32, wherein the characterization of inflammatory bowel disease comprises assessing the efficacy of treatment.

34. A method of assessing stage of cancer of a subject in a single imaging session, the method comprising:
administering a macrophage imaging agent to the subject during an administration session;
allowing a passage of time sufficient for accumulation of the agent in macrophages of the subject; and
in a single imaging session
(i) acquiring a macrophage-enhanced magnetic resonance image of regions of the subject's body at cancer risk, wherein the image is used to stage any primary cancer or metastatic cancer in the subject by assessment of macrophage density, macrophage displacement or tumor morphology associated with the cancer; and
(ii) acquiring a different magnetic resonance image targeting a physiological phenomenon other than macrophages.

35. A method according to claim 34, wherein using the image includes observing macrophage activity associated with a primary tumor or with any metastatic tumor in bone, lymph node, spleen, liver, central nervous system, lung, or other organ.

36. A method according to claim 34, wherein the regions collectively include the entire body.

37. A method according to claim 34, wherein the macrophage imaging agent is an ultrasmall superparamagnetic iron oxide particle.

38. A method according to claim 37, wherein the macrophage imaging agent is a complex of ultrasmall superparamagnetic iron oxide and a polysaccharide.

39. A method according to claim 38, wherein the polysaccharide is selected from the group consisting of dextran, reduced dextran and a derivative thereof.

40. A method according to claim 34, further comprising administering a contrast agent before acquiring the different magnetic resonance image.

41. A method according to claim 40, wherein the contrast agent is gadolinium.

42. A method of assessing efficacy of an anticancer treatment in a single imaging session with a subject, the method comprising:
administering a macrophage imaging agent to the subject during an administration session;
allowing a passage of time sufficient for accumulation of the agent in macrophages of the subject; and
in a single imaging session:
(i) acquiring a first magnetic resonance image of at least one region of the subject's body to be targeted by an anti-cancer treatment to establish a pre-treatment image, such image configured to reveal an accumulation of macrophages resulting from a pathophysiological process associated with cancer, which would not be present in the absence of such a pathophysiological process;
(ii) administering the anticancer treatment to the subject; and
(iii) acquiring a second magnetic resonance image of the at least one region of the subject's body targeted by the anticancer treatment to establish a post-treatment image, such image configured to assess an extent of any reduction in the post-treatment image compared to the pre-treatment image with respect to tumor-associated macrophage density and displacement associated with a primary cancer or metastatic cancer in the at least one region, wherein the extent of reduction in tumor-associated macrophage density and displacement is indicative of the efficacy of the anti-cancer treatment.

43. A method of assessing efficacy of an anticancer treatment according to claim 42, wherein the anticancer treatment includes a treatment selected from the group consisting of chemotherapy, extirpation, in situ ablation, radiation therapy, liposomal drug delivery, immunotherapy, gene therapy and alternative therapy.

44. A method according to claim 42, wherein the macrophage imaging agent is an ultrasmall superparamagnetic iron oxide particle.

45. A method according to claim 44, wherein the ultrasmall superparamagnetic iron oxide particle is a macrophage biomarker capable of being administered to a subject from between 12 and 168 hours prior to whole body MEMRI evaluation.

46. A method according to claim 42, wherein the at least one region is located outside of liver, spleen and lymph nodes of the subject.

47. A method of assessing inflammatory bowel disease in a single imaging session, the method comprising:
administering a macrophage imaging agent to the subject during an administration session;
allowing a passage of time sufficient for accumulation of the agent in macrophages of the subject; and
in a single imaging session
(i) acquiring a macrophage-enhanced magnetic resonance image of regions of the subject's abdomen, wherein the image is used to assess macrophage density potentially associated with a region of inflammatory bowel disease; and
(ii) acquiring a different magnetic resonance image of the potential region of inflammatory bowel disease targeting a physiological phenomenon other than macrophages.
